(19) United States
(12) Patent Application Publication
(10) Pub. No.: US 2014/0194484 A1
(43) Pub. Date: Jul. 10, 2014

(54) Method of Treating Cachexia and Sarcopenia

(71) Applicants: Andrew Coats, Abingdon (GB); John Beadle, Abingdon (GB); Stefan Anker, Abingdon (GB)

(72) Inventors: Andrew Coats, Abingdon (GB); John Beadle, Abingdon (GB); Stefan Anker, Abingdon (GB)

(21) Appl. No.: 14/101,272

(22) Filed: Dec. 9, 2013

Related U.S. Application Data
(63) Continuation-in-part of application No. PCT/GB2013/051965, filed on Jul. 24, 2013.
(60) Provisional application No. 61/675,472, filed on Jul. 25, 2012.

(51) Int. Cl. A61K 31/404

(52) U.S. Cl. CPC A61K 31/404
USPC 514/415

(57) ABSTRACT

The present invention relates to a method of treating a cachexia and/or sarcopenia with an oral dose of S-pindolol or a pharmaceutical formulation thereof and to an oral formulation for use in said method of treatment. The method and oral formulation comprise administering a total daily dose of 2.5 to 20 mg of S-pindolol or a pharmaceutical formulation comprising the same.

### CROSS-REFERENCE TO RELATED APPLICATIONS

This application is a continuation-in-part of PCT Application No. PCT/GB2013/051965 filed Jul. 24, 2013, which is related to and claims the benefit of provisional U.S. Application No. 61/675,472 filed Jul. 25, 2012. The entirety of each of these applications is hereby incorporated by reference for all purposes.

### FIELD OF THE INVENTION

The invention includes a method of treating a cachectic or sarcopenic patient with an oral dose of S-pindolol or a pharmaceutical formulation thereof and an oral formulation for use in the method of treatment.

### BACKGROUND

Currently no therapeutic agents are widely approved for the treatment of cachexia and sarcopenia.

WO 2008/068477 discloses the use of S-pindolol for the treatment of cachexia. Cachexia (from the Greek: kakos meaning bad and hexis meaning condition) is a wasting disease, associated with significant morbidity and mortality, accompanying a wide range of serious illnesses.

WO 2010/125348 discloses the use of S-pindolol in the treatment of sarcopenia. Sarcopenia is characterised by subnormal amounts of skeletal muscle which is not attributable to pro-inflammatory cytokines and may be present in people who are obese (Thomas Clinical Nutrition (2007) 26, 389-399). In particular, sarcopenia is defined as the loss of muscle mass and function which occurs in the elderly. It has been reported to occur in up to 25% of people over the age of 65 years.

In contrast, cachexia is defined as weight loss, associated with a chronic underlying disease, of at least 5% in 12 months or less. It is associated with fatigue, loss of muscle strength, a low fat free index and neuro-hormonal and biochemical abnormalities. Its pathophysiology is characterised by a negative protein and energy balance driven by a variable combination of inflammation, reduced food intake and abnormal metabolism.

Cancer cachexia occurs in about a third of all patients with cancer and has been estimated to be the direct cause of death in up to 20% of all cancer related deaths. Patients with solid tumours, colorectal cancer (CRC) and non-small cell lung cancer (NSCLC) have relatively high incidences of cachexia, approximately 28% and 34% respectively.

Pindolol is a synthetic, non-specific, beta-1 and beta-2 adrenergic receptor blocking agent (β-blocker) with intrinsic sympathomimetic activity (ISA). In addition to its β-blocking and ISA activity, pindolol is a highly potent antagonist of 5-HT1a receptors and binds to 5-HT1a receptors in the brain. It is administered as a racemic mixture of the R(+) and S(−) enantiomers, for the treatment of hypertension and angina, at doses of up to 60 mg/day.

While both its β-blocking and 5-HT1a activities reside primarily in the S(−) stereoisomer, its intrinsic sympathomimetic activity is shared equally by the R(+) and S(−) enantiomers. In addition, there is evidence of a preferential distribution of the S (−) isomer into the central nervous system. S-pindolol thus has a higher potency of both β-blockade and 5-HT1a receptor antagonism but lower ISA activity than an equivalent dose of racemic pindolol.

The present inventors have established that in the treatment of cachexia, S-pindolol shows superior efficacy to the racemic parent material, on a dose for dose basis, indicating that there is a differential efficacy that resides in the stereoisomeric form. This broadly improved activity may be due to the unique multi-functional pharmacological activity of this particular stereoisomer, which renders it optimal for use in the treatment of cachexia and sarcopenia.

In pre-clinical models of cancer cachexia some β blockers have no effect at all while some β blockers affect some aspects of the condition but not others. Of the β blockers studied, only S-pindolol had significant effects on all aspects and had the greatest impact upon survival. S-pindolol produced better effects than racemic pindolol on a dose for dose basis.

The racemic pindolol has never been marketed nor investigated clinically for the treatment of cachexia or sarcopenia. S-pindolol has never been marketed for any indication, nor has it been investigated clinically for the treatment of cachexia or sarcopenia.

Approved racemic pindolol products are administered for cardiovascular indications two to four times daily. High frequency dosing is suggested due to the short half-life of the therapeutic agent in vivo. The maximum total daily dose of racemic pindolol employed in the treatment of cardiovascular indications is 60 mg. The dose is often increased incrementally until the desired therapeutic dose is reached.

Investigational studies for the treatment of clinical depression with racemic pindolol, based upon the augmentation of Selective Serotonin Reuptake Inhibitors (SSRIs), have used doses of 2.5 mg to 5 mg pindolol three times daily, but it has been suggested that these doses may have been too low (Pindolol Augmentation of Selective Serotonin Reuptake Inhibitors: PET Evidence That the Dose Used in Clinical Trials Is Too Low. Eugenii A. Rabiner et al. Am J Psychiatry 2001; 158:2080-2082.)

In the treatment of premature ejaculation, pindolol has been used at doses of 7.5 mg once daily (Once-daily high-dose pindolol for paroxetine-refractory premature ejaculation: a double-blind, placebo-controlled and randomized study. Safarinejad M R. J Clin Psychopharmacol. 2008 Feb. 28(1):39-44).

### BRIEF DESCRIPTION OF THE DISCLOSURE

The invention includes a method of treating cachexia or sarcopenia comprising administering to human patient in need thereof a total oral dose per day of between 2.5 to 20 mg of S-pindolol.

In one embodiment the total daily dose is divided into two sub-doses and administered twice a day at different time points, for example as two equal sub-doses, for example each 1.25, 1.5, 2, 2.5, 3, 3.5, 4, 4.5, 5, 5.5, 6, 6.5, 7, 7.5, 8, 8.5, 9, 9.5 or 10 mg, such as 2.5 mg.

In another aspect there is provided a method of treating cachexia or sarcopenia comprising administering to a human patient in need thereof an oral dose of S-pindolol of 2.5 to 10 mg or a formulation comprising the same wherein the method comprises administering the dose or the formulation twice daily (bi-daily).

In another aspect there is provided an oral formulation comprising 2.5 to 10 mg of S-pindolol per dose and at least one excipient, for example characterised in that where the oral formulation is a solid dose formulation provided as a unit dose.

In another aspect there is provided a total daily dose of 2.5 to 20 mg of S-pindolol, for example for administration twice a day as a dose 2.5 to 10 mg or a formulation comprising the same for use treatment, in particular treatment of cachexia and/or sarcopenia.

In a further aspect there is provided use of S-pindolol or a pharmaceutical formulation comprising the same in the manufacture of a medicament for the treatment cachexia and/or sarcopenia by administering a total daily dose of 2.5 to 20 mg, for example wherein the dose is administered twice a day and each dose is in the range 2.5 to 10 mg.

In a further aspect there is provided a method of improving the life expectancy and/or prognosis in a cancer patient comprising administering to human patient in need thereof an oral total dose per day of between 2.5 to 20 mg of S-pindolol, for example administered twice daily as a dose in the range 2.5 to 10 mg or administered as a pharmaceutical formulation comprising the same.

1.25 to 10 mg may be a suitable alternative to 2.5 to 10 mg in a bi-daily treatment regime.

### DETAILED DESCRIPTION OF THE DISCLOSURE

The treatment regime described herein is efficacious for the treatment of cachexia and sarcopenia.

Cachexia as employed herein refers to weight loss, associated with a chronic underlying disease, for example of at least 5% in 12 months or less. It is associated with fatigue, loss of muscle strength, a low fat free index and neuro-hormonal and biochemical abnormalities. Its pathophysiology is characterised by a negative protein and energy balance driven by a variable combination of reduced food intake and abnormal metabolism.

Sarcopenia as employed herein refers to subnormal amounts of skeletal muscle related to ageing, which is not attributable to pro-inflammatory cytokines and which may be present in people who are obese or non-obese.

Thus sarcopenia is the age-associated decrease in skeletal muscle mass resulting from a variety of causes including decreased physical activity and/or decreased production of anabolic hormones. Sarcopenia is not necessarily associated with weight loss so if weight loss becomes significant, cachexia may also be present.

S-pindolol, MT-102 and espindolol are used interchangeably throughout. Treatment as employed herein refers to prophylaxis of at risk patients as well treatment following diagnosis.

Total daily dose as employed herein is the dose given in total of the active agent over the period of 24 hours.

Twice daily or bi-daily as used herein are equivalent and are intended to refer to a total daily dose divided and administered at two separate time points during the day.

In one embodiment the twice daily or bi-daily dose is administered at two separate time points during the day, such as approximately 1, 2, 3, 4, 5, 6, 7, 8, 9, 10, 11 or 12 hours apart. For example, the first dose is administered at one time point and the second dose is administered at a time point approximately 12 hours later.

An oral formulation is one which is provided in a form suitable for oral delivery, for example a solution, suspension or a solid dose for example dry granules, a tablet, capsule, caplet, sublingual or buccal formulation.

In one embodiment the formulation according to the present disclosure or employed in the present method is a solid dose formulation, for example provided as a unit.

Unit dose as employed herein refers to a discrete unit, for example a tablet, capsule, sachet, ampule or the like that contains one dose of the medicament.

Solid dose formulations may comprise pharmaceutically acceptable excipients include those independently selected from microcrystalline cellulose, lactose, sodium citrate, calcium carbonate, calcium sulphate, dibasic calcium phosphate and glycine, mannitol, pregelatinised starch, corn starch, potato starch, disintegrants such as sodium starch glycollate, croscarmellose sodium and certain complex silicates, and granulation binders such as polyvinylpyrrolidone, talc and lubricants such as magnesium stearate, stearic acid.

Capsules may be filled with a powder (of medicament alone or as blend with selected filler(s)) or alternatively a liquid, each comprising S-pindolol and a carrier. Where the capsule is filled with a powder the S-pindolol and/or the carrier may be milled or micronised to provide material with an appropriate particle size.

In one embodiment the solid dose formulations comprises one or more excipients independently selected from microcrystalline cellulose, lactose, colloidal silicon dioxide, maize starch, povidone, magnesium stearate and crospovidone.

In one embodiment the solid dose formulation is a tablet or capsule. In one embodiment the formulation is an immediate release tablet or capsule. Immediate release and instant release are used interchangeably as employed herein. Immediate-release formulation as employed herein refers to where substantially all the S-pindolol is released within a small period of time, for example 30 minutes.

In one embodiment the dosage form is an immediate release tablet or capsule containing 2.5 to 10 mg (such as 2.5 mg) of S-pindolol per dosage form.

In one embodiment the qualitative and quantitative composition for a formulation according to the present disclosure is given below in Table 1:

TABLE 1
S-Pindolol 2.5 mg tablet composition

| Component | mg/tablet | Function |
|---|---|---|
| Povidone K 30 | 3.0 | Binder |
| Ethanol* | qs | Vehicle |
| Pindolol S Isomer | 2.5 | Active |
| Lactose monohydrate (Pharmatose | 45.5 | Diluent |
| 200M) | | |
| Maize Starch (Dried) | 10.0 | Disintegrant |
| Aerosil 200 | 1.0 | Glidant/Disintegrant |
| Avicel PH 101 | 17.5 | Diluent |
| Crospovidone | 2.0 | Disintegrant |
| Avicel PH 102 | 17.5 | Diluent |
| Magnesium Stearate | 1.0 | Lubricant |

Note:
*Not present in the finished product

Formulations comprising up to 10 mg of S-pindolol can be prepared similarly as per the formulation of Table 1.

In one embodiment the solid dose formulation, such as a tablet, is rapidly disintegrating.

In one embodiment the solid dose formulation is sustained release.

For aqueous suspensions and/or elixirs, the S-pindolol may be combined with various sweetening or flavouring agents, colouring matter or dyes, with emulsifying and/or suspending agents and with diluents such as water, ethanol, propylene glycol and glycerin, and combinations thereof.

Liquid formulations may be particularly advantageous because often the elderly have difficulty swallowing solid-dose formulations.

Although the racemic pindolol product is administered three or four times daily (due to the short half-life) when used for cardiovascular indications, the present inventors have established that a bi-daily administration is particularly suitable for the treatment of cachexia and/or sarcopenia.

This novel and advantageous dosing regimen may be explained principally by the non-cardiovascular mechanism of action of S-pindolol in the treatment of cachexia and sarcopenia.

The bi-daily dosing is advantageous because it reduces the burden on the patient to take medications at various time points in the day and therefore facilitates compliance in this frail and debilitated population.

In one embodiment the cachexia is associated with an underlying disease selected from the group comprising cancer, chronic heart failure, COPD, TB, rheumatoid arthritis, cirrhosis, renal failure and HIV.

In one embodiment the cancer is selected from colorectal cancer, lung cancer, pancreatic cancer, bone cancer, stomach cancer, oesophageal cancer, prostate cancer and ovarian cancer, for example colorectal cancer and lung cancer such as non-small cell lung cancer.

In one embodiment the treatment is employed in combination with a standard cancer treatment, for example pre, post and/or concomitant with a cancer treatment regime selected from surgery, chemotherapy and radiotherapy.

Chemotherapy includes the use of antineoplastics such as alkylating agents for example cisplatin, carboplatin, oxaliplatin, mechlorethamine, cyclosphosphamide, chloroambucil, ifosamide; anti-metabolites for example purines or pyrimidines; alkaloids and terpenoids for example vinca alkaloids and taxanes such as vincristine, vinblastine, vindesine and taxol; topoisomerase inhibitors for example irinotecan, topotecan, amsacrine, etoposide, etoposide phosphate and teniposide; and cytotoxic antibiotics for example actinomycin, anthracycline, doxorubicin, danrubicin, valrubicin, idarubicin, epirubicin, bleomycin, plicamycin, mitomycin, anti-angiogenesis drugs such as bevacizumab, avastin, sutent and other more recently approved drugs such as tyrosine kinase inhibitors and 11-6 inhibitors.

In one embodiment the patient or patient population selected for treatment has a body mass index of 25 kg/m² or less, such as 24, 23, 22, 21 or 20 kg/m² or less.

In one embodiment the patient or patient population selected for treatment has a body mass index of over 25 kg/m².

In one embodiment the patient or patient population are characterised in that there has been at least 5% weight loss in the previous 12 months. Such as at least 5, 6, 7, 8, 9, 10, 11, 12, 13, 14, 15, 16, 17, 18, 19, 20, 21, 22, 23, 24, 25, 26, 27, 28, 29, 30% or above weight loss.

In one embodiment a sarcopenic patient selected for treatment has not loss overall body mass.

In one embodiment the patient or patient population are characterised wherein the patient has at least two symptoms independently selected from the group comprising subjective report of decreased muscle strength, subjective report of fatigue, subjective report of anorexia and abnormal biochemistry.

Abnormal biochemistry as employed herein refers to at least one of the following: C-Reactive Protein levels which are greater than the upper normal limit, and/or anaemia and/or low serum albumin.

Low serum albumin levels may include levels of less than 3.2 g/dl/Anaemia is a decrease in the number of red blood cells. WHO's Haemoglobin thresholds used to define anaemia:

| Age or gender group | Hb threshold (g/dl) | Hb threshold (mmol/l) |
|---|---|---|
| Children (0.5-5.0 yrs) | 11.0 | 6.8 |
| Children (5-12 yrs) | 11.5 | 7.1 |
| Teens (12-15 yrs) | 12.0 | 7.4 |
| Women, non-pregnant | 12.0 | 7.4 |
| (>15 yrs) | | |
| Women, pregnant | 11.0 | 6.8 |
| Men (>15 yrs) | 13.0 | 8.1 |

(1 g/dL = 0.6206 mmol/L)

In one embodiment the treatment is employed in combination with a treatment regime selected from the group comprising corticosteroids, anabolic steroids, p38 inhibitor, pI3 kinase inhibitor, B-Raf inhibitor and C-Raf inhibitor.

In one embodiment the treatment is employed in combination with a treatment regime selected from the group comprising corticosteroids, non-steroidal anti-inflammatories such as aspirin (acetylsalicylic acid), diflunisal, salsalate, dexibuprofen, ibuprofen, naproxen, fenoprofen, ketoprofen, dexketoprofen, flurbiprofen, oxaprozin, loxoprofen, indomethacin, tolmetin, sulindac, etodolac, ketorolac, diclofenac, nabumetone, piroxicam, meloxicam, tenoxicam, droxicam, lornoxicam, isoxicam, mefenamic acid, meclofenamic acid, flufenamic acid, tolfenamic acid, celecoxib, licofelone, lysine clonixinate, analgesics such paracetamol, anti-TNFalpha therapy, anti-IL-1 therapy, rituximab, abatacept or tocilizumab.

In one embodiment the treatment is employed in combination with a treatment regime comprising anti-retroviral therapy including combination therapy, for example combiviir, trizivir, kaletra, epzicome, truvada or atripla.

In one embodiment the treatment is employed in combination with a treatment regime comprising an antibiotic or a combination thereof, for example rifampicin, isoniazid, pyrazinamide and ethambutol.

In one embodiment the patient populations age is 50 or above, for example 55, 60, 65, 66, 67, 68, 69, 70, 71, 72, 73, 74, 75, 76, 77, 78, 79, 80, 81, 82, 83 or above.

Increasing life expectancy for a cancer patient as employed herein is intended to refer to the fact that, on average, patients administered the treatment according to the present disclosure are expected to live longer than patients who do not receive the treatment. This may translate to 2 months or more extra life expectancy, for example 3, 4, 5, 6, 7, 8, 9, 10, 11, 12 months or more increased life expectancy.

Improved prognosis as employed herein in cancer patients refers to a improvement in the health of the patient, for example improved muscle mass, less fatigue, improved ability to cope with the rigours of therapy, such as chemotherapy, reduced susceptibility to injury, improved appetite or similar.

In one embodiment the cancer is selected from colorectal cancer, lung cancer, pancreatic cancer, bone cancer, stomach cancer, oesophageal cancer, prostate cancer and ovarian cancer, for example colorectal cancer and lung cancer such as non-small cell lung cancer.

In one embodiment the cancer is selected from a tumour, colorectal cancer or lung cancer, such as non-small cell lung cancer.

In one embodiment the dose of S-pindolol does not affect blood pressure. Comprising in the context of the present specification is intended to meaning including.

In one embodiment there is provided a use of s-pindolol or a pharmaceutical formulation comprising the same in the manufacture of a medicament for the treatment of cachexia and/or sarcopenia by administering a total daily dose of 2.5 to 20 mg, for example administered twice a day as a dose of 2.5 to 10 mg.

In one embodiment a high dose is 10 mg administered bi-daily, such as a 20 mg total daily dose.

In one embodiment a low dose is 2.5 mg administered bi-daily, such as a 5 mg total daily dose.

In one embodiment a high dose promotes weight gain in a cachectic or sarcopenic patient.

In one embodiment a low dose promotes maintenance of body mass in a cachectic or sarcopenic patient.

In one embodiment treatment with S-pindolol at a high or low dose increases lean body mass.

In one embodiment treatment with S-pindolol improves or prolongs the survival of a cachectic or sarcopenic patient.

ITT, mITT and ATP analysis of data are used as per their normal meaning as understood by the skilled person.

Where technically appropriate, embodiments of the invention may be combined. Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

### BRIEF DESCRIPTION OF THE FIGURES

FIG. 1 Study Schematic

FIG. 2 Assessment Schedule for visit days 0, 28, 56, 84 and 112

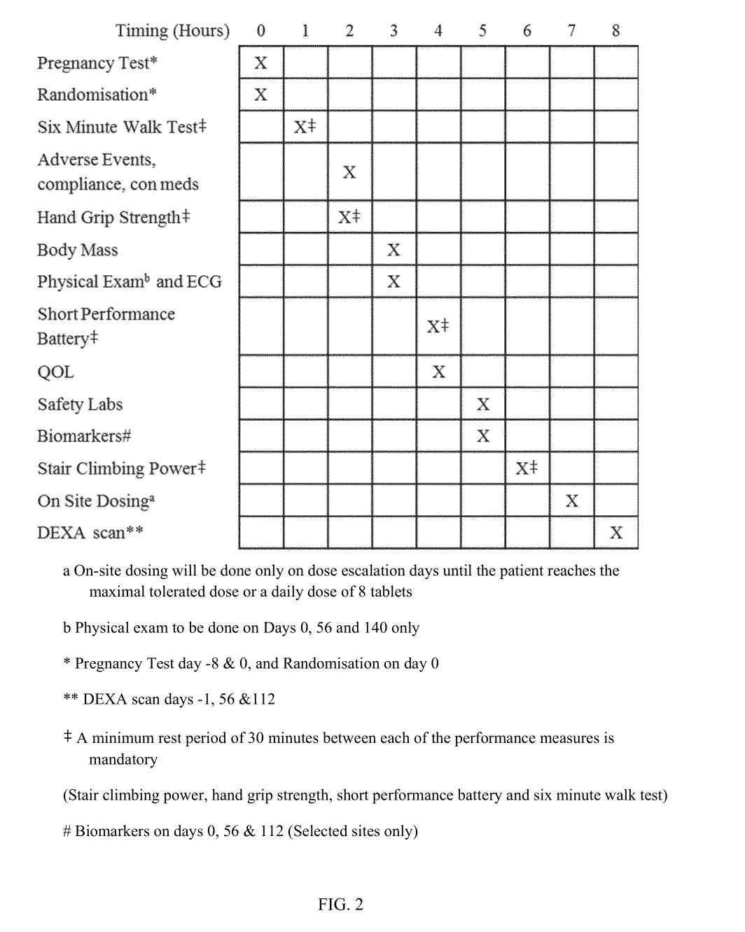

FIG. 3 Sympathetic drive in the pathogenesis of cachexia

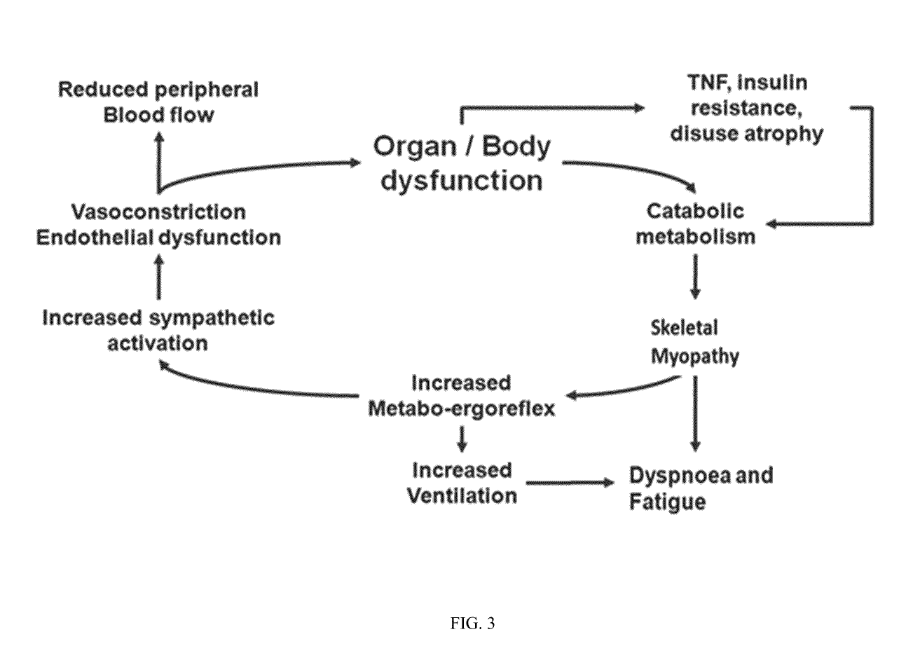

FIG. 4 S-pindolol is particularly beneficial when compared to placebo and racemic pindolol

FIG. 5 Overall survival

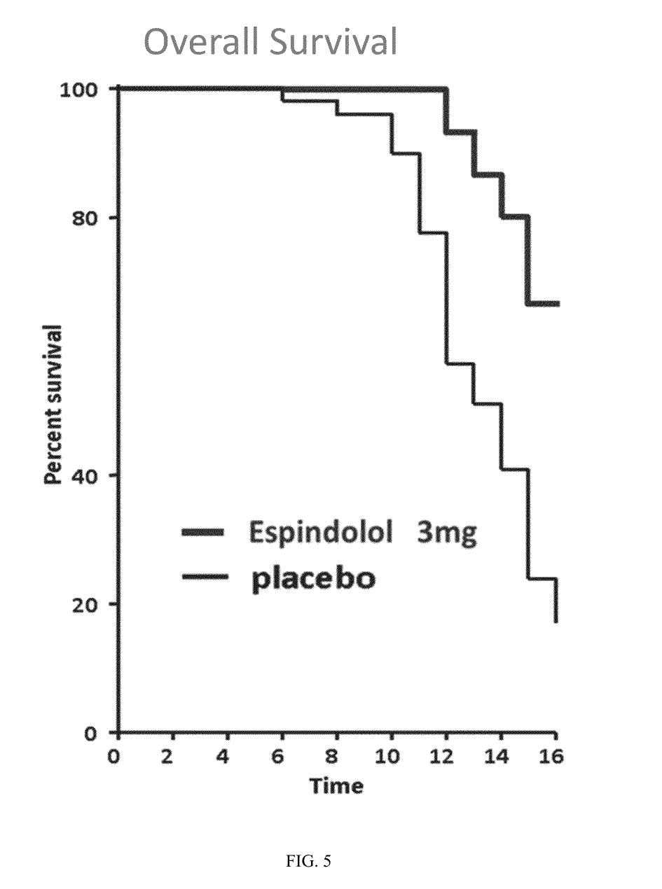

FIG. 6 Baseline characteristics (ITT)

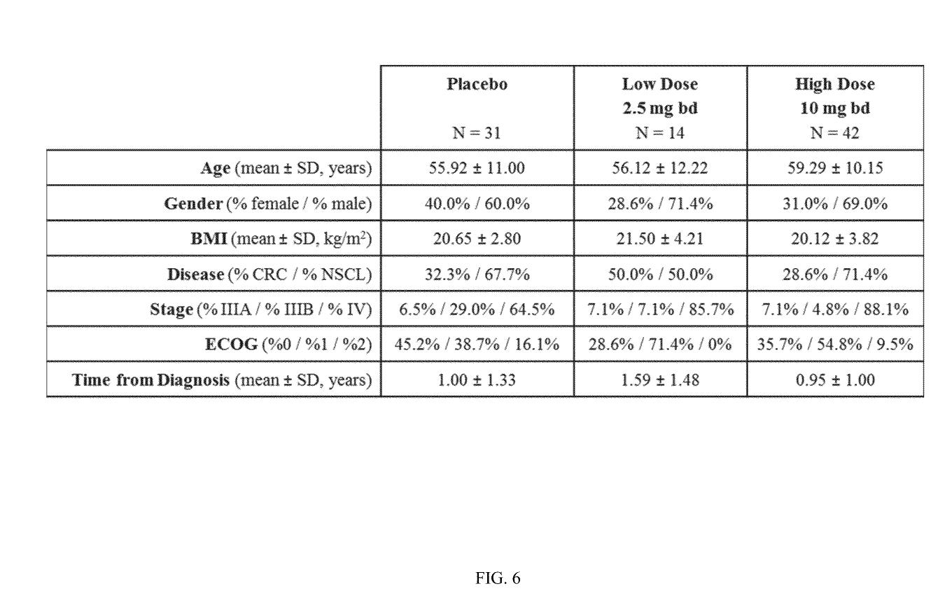

FIG. 7 Safety—All Adverse Events with Δ>5% between Arms, No significant differences in survival

FIG. 8 Safety—All Related Adverse Events—All grades

FIG. 9 Median absolute weight change

FIG. 10 Absolute weight change (ITT)

FIG. 11A shows the Body composition change (ITT) Lean Mass Change (Kg); FIG. 11B shows the Body composition change (ITT) Fat Mass Change (Kg)

FIG. 12 Functional data (ITT)—Slopes of change—Derived using the Mixed-Effect Model for Repeated Measures

FIG. 13 Relative change of weight by visit (ITT Population)

FIG. 14 Slope of weight change by patient (ITT Population)

FIG. 15 Slope of weight change by patient (ITT Population)*

FIG. 16 Weight change (%) over time by patient—Patients on Low-Dose MT-102 (ITT Population)

FIG. 17 Weight change (%) over time by patient—Patients on High-Dose MT-102 (ITT Population)

FIG. 18 Weight change (%) over time by patient—Patients on Placebo (ITT Population)

FIG. 19 Relative change of weight by visit (mITT Population)

FIG. 20 Slope of weight change by patient (mITT Population)

FIG. 21 Weight change (%) over time by patient—Patients on Low-Dose MT-102 (mITT Population)

FIG. 22 Weight change (%) over time by patient—Patients on High-Dose MT-102 (mITT Population)

FIG. 23 Weight change (%) over time by patient—Patients on Placebo (mITT Population)

FIG. 24 Relative change of weight by visit (ATP Population)

FIG. 25 Slope of weight change by patient (ATP Population)

FIG. 26 Weight change (%) over time by patient—Patients on Low-Dose MT-102 (ATP Population)

FIG. 27 Weight change (%) over time by patient—Patients on High-Dose MT-102(ATP Population)

FIG. 28 Weight change (%) over time by patient—Patients on Placebo (ATP Population)

### EXAMPLES

### Study Design and Conduct

An international multicentre, randomised, double blind, placebo controlled clinical trial was conducted with three parallel treatment groups with dose escalation within each group. Male and female patients aged between 25 to 80 years of age with cachexia related to underlying progressive or recurrent late stage colorectal cancer (CRC) or non-small cell lung cancer (NSCLC) were treated for 16 weeks with either S-pindolol or placebo. All patients received standard of care chemotherapy, radiotherapy and supportive care throughout the study at the discretion of their treating physician. The physician took the necessary judgment and planned the chemotherapy cycles and the functional assessment visits in such a manner that chemotherapy does not interfere with the performance of the functional assessments by the patients.

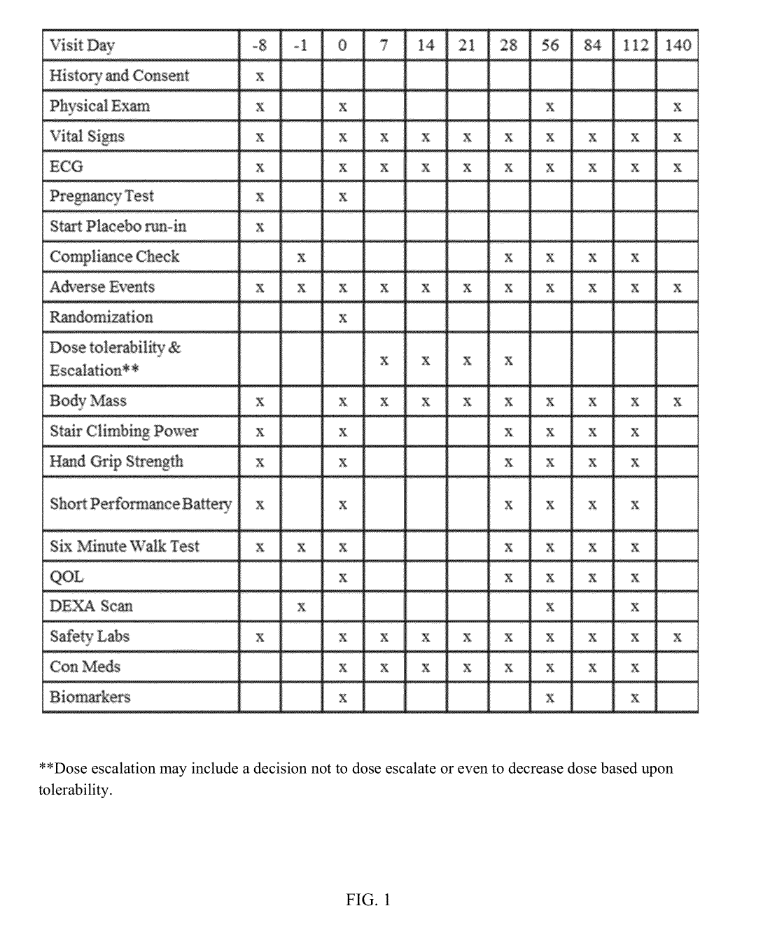

After provision of written informed consent, patients were assessed for eligibility at screening visits occurring from days -8 to -1. All eligible patients with written informed consent were given a set of performance tests [stair climbing power (SCP), hand grip strength (HGS), short performance battery test (SPBT) and the six minute walk test (SMWT)] and receive a 7 day course of placebo (1 tablet two times per day). Patients able to adequately complete the performance tests and with at least 80% compliance after 7 days (tablet count: at least 11 out of 14 tablets) were then randomised on Day 0 to one of three treatment groups. All patients attended for visits on days 7, 14, 21, 28, (each +/−1 day), 56, 84, 112, and 140 (each +/−7 days). The schedule of visits, examinations and investigations is summarised in the Study Schematic shown in FIG. 1.

The dose of S-pindolol or placebo was escalated by the investigator in a blinded fashion as described below under the heading “Dose Escalation”. The maximum period of drug titration was 4 weeks and the minimum period was 2 weeks. The patients were required to attend study visits at 1 week intervals after the start of the titration phase until completion of dose escalation. The aim of dose escalation for all subjects is to reach the target dose of 8 tablets per day (4 tablets bd) or the maximum tolerated dose for a particular patient within the first 4 week period. Dose titration was achieved by varying the number of tablets taken from bottle 2. This was done such that patients will be randomised to receive either placebo, 2.5 mg S-pindolol twice daily, 5 mg S-pindolol twice daily, 7.5 mg S-pindolol twice daily, or 10 mg S-pindolol twice daily according to tolerance of the study drug.

### Subject Selection

### Inclusion Criteria:

1. Adult patients aged between 25 to 80 years of age and with a life expectancy of greater than 3 months as judged by the treating physician.
2. Confirmed diagnosis of one of:
   a. Non-curative stage III or stage IV Colorectal Cancer (CRC) not suitable for surgery, or
   b. Non-curative stage III or stage IV Non-small Cell Lung Cancer (NSCLC) not suitable for surgery;
3. Patients who are receiving or who have already received a course of chemotherapy, with or without radiotherapy or surgery, with one of the following regimes:
   a. For non-small cell lung cancer, a platinum based regimen
   b. For colorectal cancer, a 5FU or Irinotecan based regimen
4. Cachexia with ongoing weight loss that in the opinion of the investigator is due to the underlying cancer.
5. Evidence of cachexia as judged by one of:
   a. >5% documented weight loss in the previous 12 months; or
   b. A subjective report of weight loss in the previous 12 months and a recorded body mass index (BMI) less than 20.0 kg/m²
   c. Ongoing documented weight loss of at least 1kg in the week prior to day 0; or 1.25kg in the 2 weeks prior to day 0, or 1.5kg in the 3 to 6 weeks prior to day 0; provided that BMI is not more than 25.
6. At least two of the following:
   a. Subjective report of decreased muscle strength
   b. Subjective report of fatigue
   c. Subjective report of anorexia
   d. Abnormal biochemistry with one or more of the following:
      i. CRP >ULN (as per Central Lab normal value)
      ii. Anemia (<12 g/dl)
      iii. Low serum albumin (<3.2 g/dl)
7. Patients of childbearing potential must use an effective method of avoiding pregnancy (including oral, transdermal, or implanted contraceptives; an intrauterine device; male or female condoms; diaphragm or cervical cap with spermicide; or abstinence) prior to randomisation and must agree to continue using such precautions until the end of the 140 day safety follow up;
8. Willing and able to comply with the protocol and to complete the study period;
9. Willing to forego other forms of experimental treatment during the study;
10. Signed and dated informed consent, prior to receipt of any study medication or any study related procedures.
11. ECOG performance status 0, 1 or 2.
12. Able to complete the performance tests (SCP, SMWT, SPPB, HGS) at the screening visit and with two consecutive pre-randomisation SMWT results that differ by no more than 30% from each other.
13. At least 80% compliant during the placebo run in period.

### Exclusion Criteria:

1. Pregnancy or lactation at screen or baseline visit;
2. >20% weight loss in the previous 3 months or a BMI of less than 16 kg/m²
3. Age greater than 80 or less than 25 at baseline visit;
4. Scheduled to start any new course of chemotherapy or to undergo a change in present chemotherapeutic regimen during the dose escalation phase of the study (the first three weeks after randomisation);
5. Any surgical procedure within the past month or any planned surgical procedure;
6. Any mechanical obstruction of the alimentary canal;
7. Any history or evidence of intractable vomiting;
8. A history or clinical evidence of any hyperthyroidism, cirrhosis, hepatic failure, HIV, renal failure (as determined by a serum creatinine >250 μmol/l or >2.83 mg/dl at screen) or active tuberculosis (as confirmed by sputum or other microbiological methods, within the last five years);
9. Any physical, medical, socioeconomic or other non-cancer related cause for simple starvation, muscle wasting or weight loss;
10. Receiving enteral tube feeding or parenteral nutrition at screening or baseline visit;
11. Any clinical evidence of ascites or significant oedema or significant pleural effusion at screening or baseline visit;
12. Current or planned treatment with
   a. Any oral adrenal corticosteroids (inhaled or topical steroids and short-term use of dexamethasone around the time of chemotherapy are acceptable);
   b. Beta adrenergic blockers,
   c. Non-dihydropyridine calcium antagonists (e.g. Verapamil, diltiazem),
   d. Alpha adrenergic blockers,
   e. Ivabradine (Coralan, Procoralan),
   f. 5HT agonists or antagonists e.g. SSRI's, (short-term use around the time of chemotherapy are acceptable)
   g. MAOI's,
   h. Beta agonists, (short term or on-and -off use of inhaled broncho-dilators are acceptable)
   i. Amiodarone, and
   j. Megestrol, Anabolic Steroids or any other prescription medication intended to increase appetite or to treat unintentional weight loss.
13. Treatment with any investigational drug therapy within 28 days prior to the screening visit;
14. Previous history of administration of pindolol or S-pindolol;
15. History of allergy or reaction to any component of the MT 102/study drug formulation;
16. History or presence of congestive heart failure (with LVEF <45%) or uncontrolled hypertension (with BP >160/95 mm Hg);
17. Use of a pacemaker, implantable defibrillator, or internalized metal stent;
18. Resting pulse rate less than 68 beats per minute or high degree conduction defect on the electrocardiogram;
19. A resting supine systolic blood pressure less than 100 mm Hg.
20. A history of bronchospasm and bronchial asthma;
21. History or diagnosis of brain metastases
S-pindolol Supplies:
S-pindolol: S-pindolol was provided as 2.5 mg immediate release tablets.
Placebo: Placebo was supplied as tablets matching the S-pindolol tablets. The placebo formulation was identical to the drug product in all respects, with the exception of the active substance.
All tablets were packaged in bottles. Patients received two bottles, Bottle “1” and Bottle “2”. Bottle 1 contained one month dosing of either 2.5 mg active or identical placebo tablets, which patients take throughout the trial. Bottle “2” contained one month supply either 2.5 mg active or identical placebo tablets which were used to up or down titrate the dose following Investigators review of tolerability of current dose. Packages were labelled in accordance with local regulatory requirements.

### Treatment Regimens

S-pindolol or placebo was administered over a 16 week period. The treatment regimens for the study are summarised below.
At regular monitoring visits, a study monitor checked that all code-break envelopes remained intact. The monitor remained blind during the study, and steps were taken during monitoring such that treatment allocation was not revealed, including patients for whom the envelope was opened.

TABLE 2
Treatment Regimens

| | Patient | Daily | | |
|---|---|---|---|---|
| Group | No's | Dose | Day No's | Treatment* |
| A | 22 | 5 mg | 0-6 | 1 × 2.5 mg tablet of S-pindolol two times per day |
| | | 5 mg | 7-13 | 1 × 2.5 mg tablet of S-pindolol + 1 placebo tablet two |
| | | | | times per day |
| | | 5 mg | 14 onwards | 1 × 2.5 mg tablet of S-pindolol + 3 placebo tablet two |
| | | | | times per day |
| B | 66 | 5 mg | 0-6 | 1 × 2.5 mg tablet of S-pindolol two times per day |
| | | 10 mg | 7-13 | 2 × 2.5 mg tablet of S-pindolol two times per day |
| | | 20 mg | 14 onwards | 4 × 2.5 mg tablets of S-pindolol two times per day |
| C | 44 | 0 mg | 0-6 | 1 × placebo tablet two times per day |
| | | 0 mg | 7-13 | 2 × placebo tablet two times per day |
| | | 0 mg | 14 onwards | 4 × placebo tablet two times per day |

*Patients who were unable to tolerate the dose escalation in any group were allowed to decrease their dose under the investigator's supervision, without breaking the blind.

### Administration of Investigational Product

Product was taken orally with food as prescribed two times daily

### Concomitant Medications

Disallowed Medications:
   a. The following medications are not permitted during the study: Any oral adrenal corticosteroids (inhaled or topical steroids and short-term use of dexamethasone around the time of chemotherapy are acceptable);
   b. Beta adrenergic blockers,
   c. Non-dihydropyridine calcium antagonists (eg Verapamil, diltiazem),
   d. Alpha adrenergic blockers,
   e. Ivabradine (Coralan, Procoralan)
   f. 5HT agonists or antagonists e.g. SSRI's, (short-term use around the time of chemotherapy are acceptable)
   g. MAOI's,
   h. Beta agonists, (short term or on-and -off use of inhaled broncho-dilators are acceptable)
   i. Amiodarone,
   j. Megestrol (Megace), anabolic steroids or any other prescription medication intended to increase appetite or to treat unintentional weight loss

### Screening Visit Day-8

Assessments conducted between days-8 days to -1:
1. History and Consent
2. Verify eligibility criteria
3. Physical Examination
4. Vital signs
5. ECG
6. Pregnancy test as applicable—Urine or serum β-HCG
7. Weight, height and derived BMI
8. Blood and urine samples for safety
9. Conduct the following assessments:
   a. the Short Physical Performance Battery test(SPPB)
   b. the six minute walk test (SMWT)
   c. hand grip strength (HGS)
   d. Stair climbing power (SCP)
10. Provided placebo for compliance test. Patients were given a single bottle of tablets and told that this is the start of their treatment. They were instructed to take 1 tablet twice a day for seven days and to return at the end of the seven day period. On the return visit, the investigator counted the remaining tablets to establish if the patient had been compliant. Only patients achieving at least 80% compliance during this period were randomised.
   Study Day 0: First administration of study drug
Assessments conducted on Study Day 0:
1. Conduct compliance check (if not done already on Day -1)
2. Pregnancy test as applicable—Urine β-HCG
3. Record concomitant medications
4. Verify eligibility criteria
5. Randomisation and assignment of investigational product number
6. Conduct the six minute walk test
7. Conduct hand grip strength (HGS)
8. Physical examination
9. Vital signs
10. ECG
11. Weight, and derived BMI
12. Conduct the Short Physical Performance Battery test (SPPB)
13. Administer QoL instrument
14. Conduct Stair climbing Power (SCP)
15. Blood and urine samples for safety
16. Blood samples for biomarkers (selected sites only)
17. Administration of investigational product as a test dose
18. Assess for new AEs, SAEs and protocol-related AEs
19. Conduct post-dose 2 hour assessment of tolerability
   Study Day 7±1 day: First dose escalation
Assessments conducted on Study Day 7:
1. Record AE's, SAEs and protocol-related AEs
2. Update concomitant medications
3. Weight and derived BMI
4. Vital signs
5. ECG
6. Blood and urine samples for safety
7. Administration of test dose according to dose escalation schedule
8. 2 hour assessment of tolerability
9. Make dose escalation decision and advise patient of new dose schedule
   Study Day 14±1 day: Second dose escalation.
Assessments conducted on Study Day 14:
1. Record AE's, SAEs and protocol-related AEs
2. Update concomitant medications
3. Weight and derived BMI
4. Vital signs
5. ECG
6. Blood and urine samples for safety
7. Administration of test dose according to dose escalation schedule
8. 2 hour assessment of tolerability
9. Make dose escalation decision and advise patient of new dose schedule
   Study Day 21±1 day: Third dose escalation
Assessments conducted on Study Day 21:
1. Record AE's, SAEs and protocol-related AEs
2. Update concomitant medications
3. Weight and derived BMI
4. Vital signs
5. ECG
6. Blood and urine samples for safety
7. If required, administration of test dose according to dose escalation schedule
8. If required, 2 hour assessment of tolerability
9. Review patient tolerability and advise patient of any alteration to dose schedule

### Study Day 28±1 day: Assessment Visit One

1. Conduct the six minute walk test (SMWT)
2. Record AE's, SAEs and protocol-related AEs
3. Compliance Check
4. Update concomitant medications
5. Conduct hand grip strength (HGS)
6. Weight, and derived BMI
7. Vital signs and ECG
8. Conduct the Short Physical Performance Battery test(SPPB)
9. Conduct measures of quality of life (QOL)
10. Blood and urine samples for safety
11. Conduct the stair climbing power (SCP)
12. If required, administration of test dose according to dose escalation schedule
13. If required, 2 hour assessment of tolerability
14. Review patient tolerability and advise patient of any alteration to dose schedule
   Study Day 56±7 days: Assessment Visit Two
1. Conduct the six minute walk test (SMWT)
2. Record AE's, SAEs and protocol-related AEs
3. Compliance Check
4. Update concomitant medications
5. Conduct hand grip strength (HGS)
6. Weight, and derived BMI
7. Physical examination; Vital signs and ECG
8. Conduct the Short Physical Performance Battery test(SPPB)
9. Conduct measures of quality of life (QOL)
10. Blood and urine samples for safety
11. Blood samples for biomarkers (selected sites only)
12. Conduct the stair climbing power (SCP)
13. Body composition according to Dual Energy X-ray Absorbitometry (DEXA)
   Study Day 84±7 days: Assessment Visit Three
1. Conduct the six minute walk test (SMWT)
2. Record AE's, SAEs and protocol-related AEs
3. Compliance Check
4. Update concomitant medications
5. Conduct hand grip strength (HGS)
6. Weight, and derived BMI
7. Vital signs and ECG
8. Conduct the Short Physical Performance Battery test(SPPB)
9. Conduct measures of quality of life (QOL)
10. Blood and urine samples for safety
11. Conduct the stair climbing power (SCP)
   Study Day 112±7 days: End of dosing visit
1. Conduct the six minute walk test (SMWT)
2. Record AE's, SAEs and protocol-related AEs
3. Compliance Check
4. Update concomitant medications
5. Conduct hand grip strength (HGS)
6. Weight, and derived BMI
7. Vital signs and ECG
8. Conduct the Short Physical Performance Battery test(SPPB)
9. Conduct measures of quality of life (QOL)
10. Blood and urine samples for safety
11. Blood samples for biomarkers (selected sites only)
12. Conduct the stair climbing power (SCP)
13. Body composition according to Dual Energy X-ray Absorbitometry (DEXA)
   Study Day 140±7 days: Follow-up Visit
1. Record AE's, SAEs and protocol-related AEs
2. Weight, and derived BMI
3. Physical examination; Vital signs and ECG
4. Blood and urine samples for safety

### Biomarker Tests

Samples were stored at -70 degrees centigrade and then analysed after completion of the study. A panel of nutritional, inflammatory cardiovascular and neuro-hormonal biomarkers were conducted on these retained samples. Depending upon the clinical outcomes observed in this study the constituents of this panel were selected from: total cholesterol, low density lipoprotein, high density lipoprotein, triglycerides, pre-albumin, cortisol, high-sensitivity C-reactive protein, interleukin-6, tumour necrosis factor (TNF), soluble TNF-receptors, pro-calcitonin, adrenaline, nor-adrenaline, aldosterone, cortisol, hehydroepiandrosterone, free testosterone, matrix metalloproteinase-2,matrix metalloproteinase-9, growth hormone, insulin-like growth factor-1, leptin, allantoin, B-type natriuretic peptide (BNP), N-terminal BNP, atrial natriuretic peptide (ANP), mid-region pro-ANP, co-peptin, pro-adrenomedulin and s-pindolol. The biomarker panel was used to examine for potential correlates of safety and efficacy for future clinical examination and research.

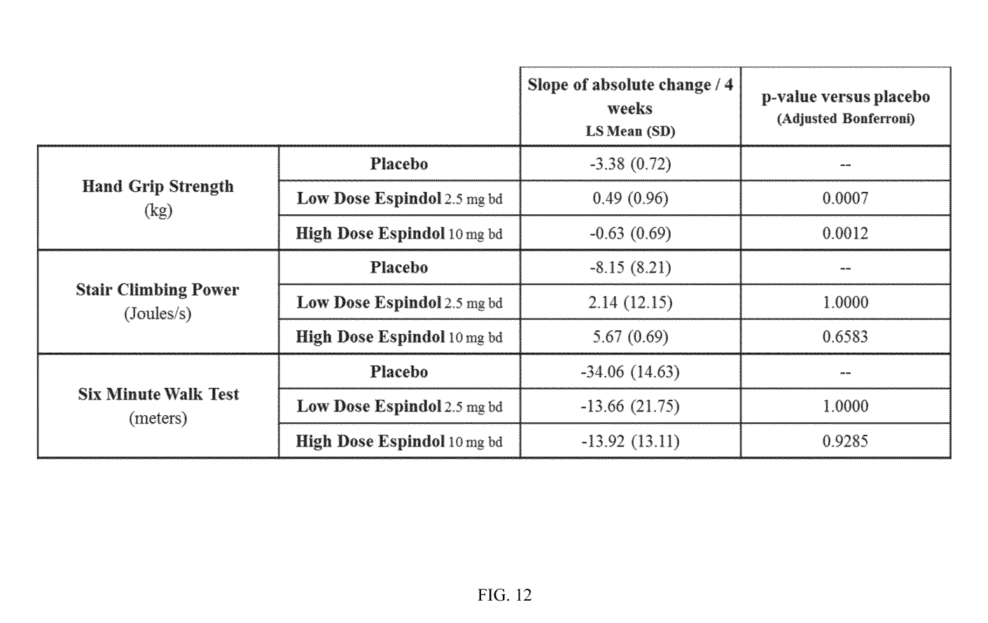

### Functional Evaluations (See FIG. 12)

### Six Minute Walk Test (SMWT)

A SMWT was conducted on days 0, 28, 56, 84 and 112. The SMWT was conducted as described by Paul L Enright (Respiratory Care, August 2003 Vol 48 No 8)¹². A straight, level and even area at least 20 meter long and 2 meters wide was marked out at each investigational site and used for the SMWT for all subjects at each time point. Identical chairs were placed at each end of the 20 meter track.

The patient was well rested prior to the test and must not have undergone any physical exertion for at least thirty (30) minutes. If required, the patient was transported to the site of the test by wheelchair and / or elevator.

The investigator could not walk with the patient and could not assist the patient. The patient had to walk alone, not with other patients, relatives or carers. The investigator should use standardized phrases while speaking to the patient using a standard script in the patient's preferred language. No relatives or carers gave instructions, assistance or encouragement during the test. Using the script, the investigator instructed the patient to walk from end to end of the track for a total of six (6) minutes using a self-elected pace but to cover as much distance as they can during the six minutes. Patientscould stop and rest at any time, but were to be instructed to resume walking as soon as they felt able to do so using the standard script. An assistant was present during this test and keep a record of the time and the number of lengths completed. The assistant called out the time in 2 minute intervals.

After the 6 minutes elapse, the total distance walked was measured to the nearest meter. If the patient is physically unable to walk at all, then a reason was recorded in the CRF and the distance walked entered as zero.

### Stair Climbing Power (SCP)

The SCP test was conducted on days 0, 28, 56, 84 and 112. The SCP test was modified from that described by Bean et al (Arch Phys Med Rehabil Vol 88, May 2007) to account for the different stairway configurations at the investigational sites.

At each investigational site, a regular, straight and even flight of seven (7) stairs was marked out and used for the SCP test for all subjects at each time point. Each step was as close as possible to 15.2 cms high, but no less than 14 cms and no more than 17.8 cms in height. The stairway was well lit and clear of any people throughout the test. The site made a note of the height of the steps; the height was measured in centimetres.

The patient was rested prior to the test and not have undergone any physical exertion for at least thirty (30) minutes before hand. If required, the patient was transported to the site of the test by wheelchair and / or elevator.

The patient was asked to climb as quickly as possible from the bottom of the stairs to the top of the steps or until the instructions to stop climbing are given by the site staff The test started when the patient begins to climb and the stop watch was stopped when the patient had both their feet on the seventh step. The investigator should instruct the patient using a pre-specified script in the patient's preferred language. No relatives or carers gave instructions, assistance or encouragement during the test and no one other than the patient should climb the stairs with the patient. If required the patient could rest and / or use the banister or wall for support, but the investigator encouraged them to start again as soon as possible. An assistant counted the steps climbed and recorded the time to complete the test using a stopwatch to the nearest 0.01 second. If the patient was unable to reach the seventh step within four minutes, then the time was recorded as four minutes and the actual height climbed (in centimetres) in that time recorded. After a fifteen minute rest, all subjects were asked to repeat and the final result of the test will be the best Power / Kg achieved across the two repeats.

The site noted the total height climbed in centimetres up to one decimal point and the time taken up to centiseconds and record the values on the CRF.

If the patient was physically unable to climb any steps at all, then a reason was recorded in the CRF and the time recorded as four minutes and the distance climbed should be entered as zero.

Power (P) was calculated as force multiplied velocity. For Stair Climbing Power the Force was the patient's body mass (m) multiplied by the acceleration due to gravity (g); and the velocity was the vertical height climbed (h) divided by the time taken (t) such that:

SCP=(m×g)×(h÷t)

Power is a derived value and will not be recorded in the CRF. Results will be analysed as a normalised Power / kg according to the methods of Bean et al.

Short Physical Performance Battery (SPPB) test

A SPPB test was conducted on days 0, 28, 56, 84 and 112. The SPPB test was conducted as described by Guralnik et al (Journal of Gerontology 1994, Vol 49, No 2). The test involved an assessment of standing balance, the timed 4.0-m walk, and a timed test of 5 repetitions of rising from a chair and sitting down. All times were measured to the nearest .01 second with a stopwatch. Each of these sub-tests was scored between 0 and 4 and summed to a maximum score of 12.

The test was conducted using the instructions as downloaded from the relevant NIH website (www.grc.nia.nih.gov/branches/ledb/sppb/index.htm). The investigator used the pre-specified script in the patient's preferred language and an assistant counted and recorded the results. No relatives or carers gave instructions, assistance or encouragement.

### Hand Grip Strength (HGS) Test

A HGS test was conducted on days 0, 28, 56, 84 and 112. The HGS test was modified from that described by Bassey (1990; NIH, 1990). A standard handgrip dynamometer (provided by Sponsor) that can be adjusted for hand size was used at all sites. Both hands were alternately measured in triplicate, followed by a fourth attempt on the dominant hand only. After an explanation the subject squeezed the handle as forcefully as possible for a few seconds and then released. Subjects were encouraged verbally using a standard script and the best score (in kilograms) of the dominant hand recorded in the CRF.

### Quality of Life (QoL)

QoL was assessed using the EQ-5D instrument on days at day 0, 28, 56, 84 and 112. The EQ-5D questionnaire was administered by the investigator using the relevant validated instrument and using the patients preferred language. The patient's answers were recorded directly into the CRF by the investigator. The patient was asked to complete the Visual Analog Scale (VAS) section of the EQ-5D directly on the CRF.

### Dual Energy X-ray Absorbitometry (DEXA)

DEXA was performed at selected DEXA scan centres on days at day -1, 56 and 112.

### Dose Escalation

The dose of S-pindolol was escalated by the investigator in a blinded fashion.

The maximum period of drug titration was 4 weeks and the minimum period was 2 weeks. The Patients were required to attend study visits at 1 week intervals after the start of the titration phase until completion of dose escalation. The aim of dose escalation was to reach the target dose of 8 tablets per day (4 tablets bd) or the maximum tolerated dose for a particular patient within the first 4 week period.

The investigator increased the dose as scheduled unless the patient had symptoms of intolerance as described below, such that the dose was not escalated if, two hours after a given test dose:
a. The resting heart rate is <50 beats per min,
b. The supine systolic blood pressure is <90 mmHg,
c. The systolic blood pressure drops >30 mmHg on assuming a sitting position
d. There are any significant new symptoms of postural hypotension or dizziness which interfere with daily activities
e. The maximum dose (8 tablets per day given as 4 tablets bd) has been achieved
f. The four week dose escalation period is completed.

During the first four weeks, the investigator increased or decreased the dose received by the patient in order to achieve the maximum tolerated dose according to criteria a) to f) above. If a given dose increase was not tolerated then the investigator attempted to increase the dose in the subsequent week unless signs of intolerance persist. If signs of intolerance persisted despite no dose escalation then the Investigator could decrease the dose. The dose was titrated by varying the number of tablets taken from bottle 2. All patients always took 1 tablet bd from Bottle 1 throughout the study unless discontinued.

The first dose of any altered regimen was administered as a test dose by the investigator and the patient was monitored for adverse reactions for at least 2 h following administration. The dose escalation was achieved as follows:
Day 0: Test dose: one tablet from bottle 1 (dose schedule 1)
   If tolerated then continue dosing with one tablet from bottle 1 bd (two tablets daily in total)
   If not tolerated then discontinue from the study and replace the patient
Day 7 (+/−1 day): Test dose: one tablet from bottle 1 and one tablet from bottle 2 (dose schedule 2)
   If tolerated: then escalate dose to one tablet each from bottle 1 bd and one tablet from bottle 2 bd (four tablets daily in total)
   If not tolerated: then maintain previous dose (one tablet from bottle 1 bd) and attempt dose escalation again at day 14.
Day 14 (+/−1 day): If previous dose escalation was tolerated then:
   Test dose: one tablet from bottle 1 and three tablets from bottle 2(dose schedule 3)
   If tolerated: then escalate dose to one tablet from bottle 1 bd and three tablets from bottle 2 bd (eight tablets daily in total)
   If not tolerated: then maintain on dose schedule 2 and attempt dose escalation again at day 21.
Day 14 (+/−1 day): If previous dose escalation was not tolerated then:
   Test dose: one tablet from bottle 1 and one tablet from bottle 2 (dose schedule 2)
   If tolerated: then escalate dose to one tablet each from bottle 1 and bottle 2 (four tablets daily in total=dose schedule 2)
   If not tolerated: then maintain dose schedule 1 and attempt dose escalation again at day 21.
Day 21 (+/−1 day) If maximum dose (dose schedule 3=eight tablets daily in total) is well tolerated then: maintain on that dose.
Day 21 (+/−1 day) If maximum dose has not yet been achieved or is poorly tolerated then: test dose with either dose schedule 1, 2 or 3 as appropriate and then maintain on the maximum tolerated dose.
Day 28 (+/−1 day) If maximum dose (dose schedule 3=eight tablets daily in total) is well tolerated then: maintain on that dose.
Day 28 (+/−1 day) If maximum dose has not yet been achieved or is poorly tolerated then: test dose with either dose schedule 1, 2 or 3 as appropriate and then maintain on the maximum tolerated dose.

### Results

There were a total of 88 patients in BBDD. One of them was not randomized so it has been considered as screening failure. The study populations have been analysed excluding this case.

TABLE 3
Analysis populations' distribution.

| | | Low dose | | |
|---|---|---|---|---|
| | | MT-102 | High dose | MT-102 Placebo |
| | | (N = 14) | (N = 42) | (N = 31) |
| Safety | | | | |
| Yes | N (%) | 14 (100.00) | 42 (100.00) | 31 (100.00) |
| ITT | | | | |
| Yes | N (%) | 14 (100.00) | 42 (100.00) | 31 (100.00) |
| mITT | | | | |
| Yes | N (%) | 11 (78.57) | 30 (71.43) | 25 (80.65) |
| No | N (%) | 3 (21.43) | 12 (28.57) | 6 (19.35) |
| ATP | | | | |
| Yes | N (%) | 11 (78.57) | 29 (69.05) | 25 (80.65) |
| No | N (%) | 3 (21.43) | 13 (30.95) | 6 (19.35) |

NOTE: Patients with early withdrawal before visit 7 for whom the study drug accountability section at CRF has not been reported have not been included in the mITT as the treatment compliance has not been calculated. However, there were three patients who withdrew before visit 7 and have reported the study drug accountability in Visit 10 or Visit 7. To be consistent with the remaining similar cases, treatment compliance was not calculated for these 3 subjects, so none of them are included in the mITT population.

### Statistical Methods

Imputation of missing data for slope calculations:
Step 1: Patients who died or discontinued due to AEs or death before 4 weeks:

Missing data imputed using the worst slope in the whole study
Step 2: all remaining cases:

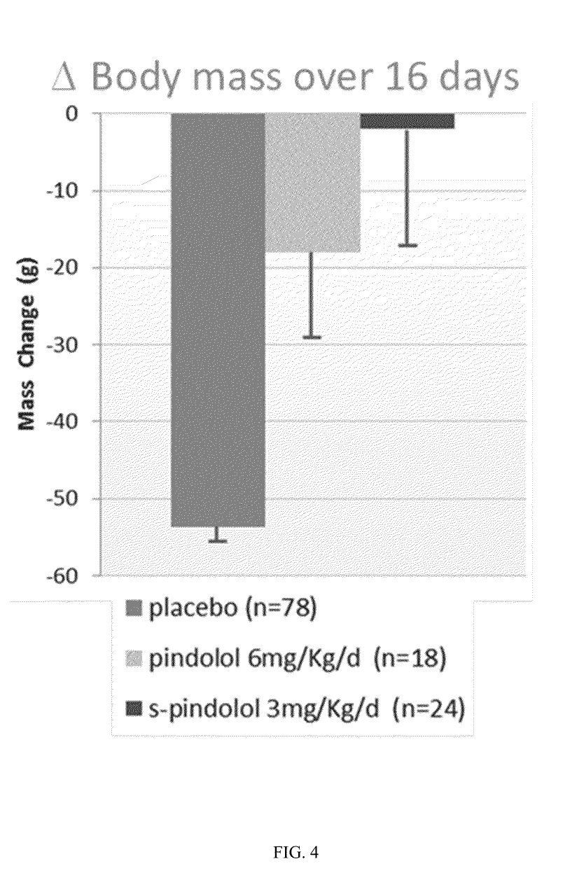

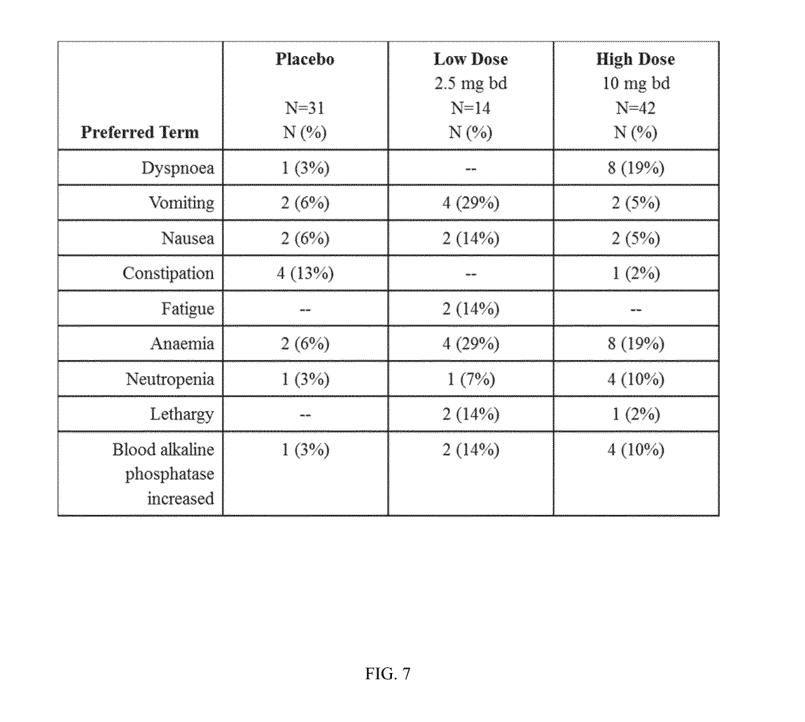

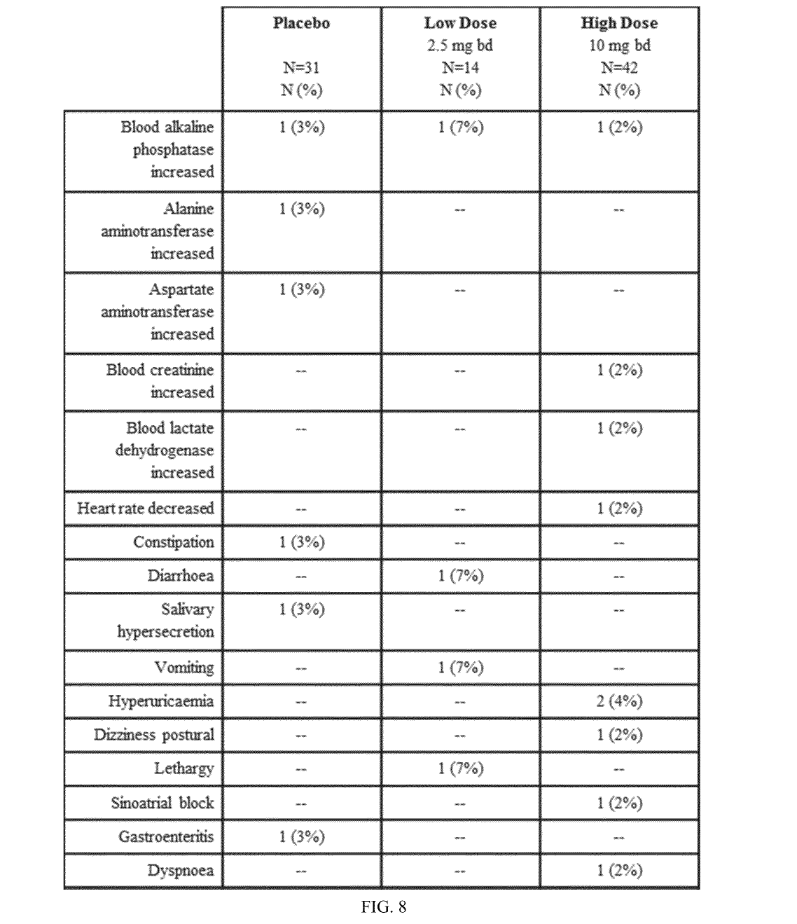

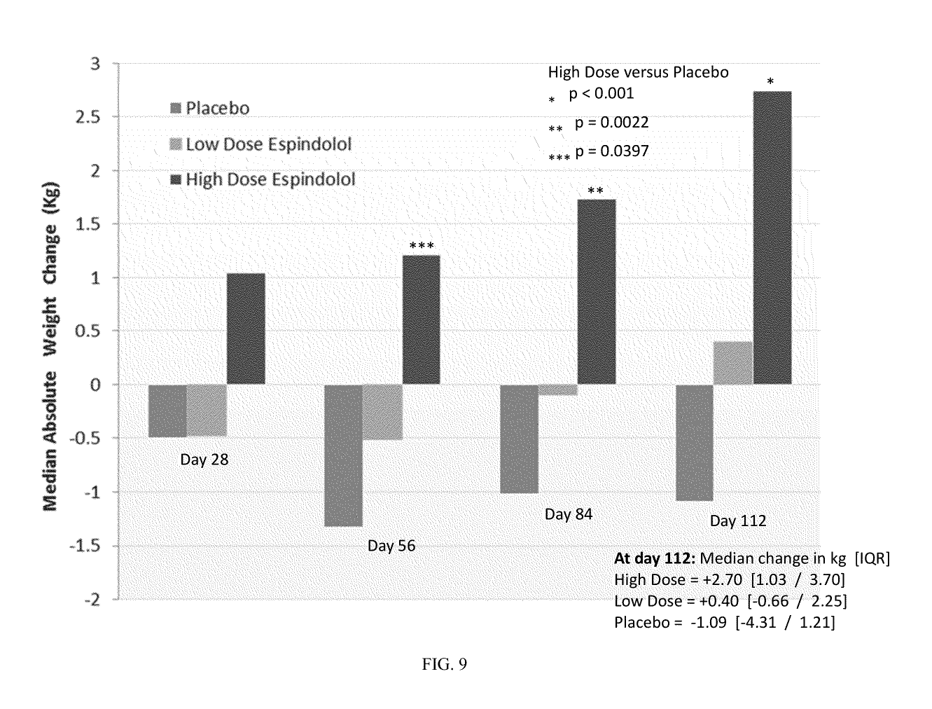

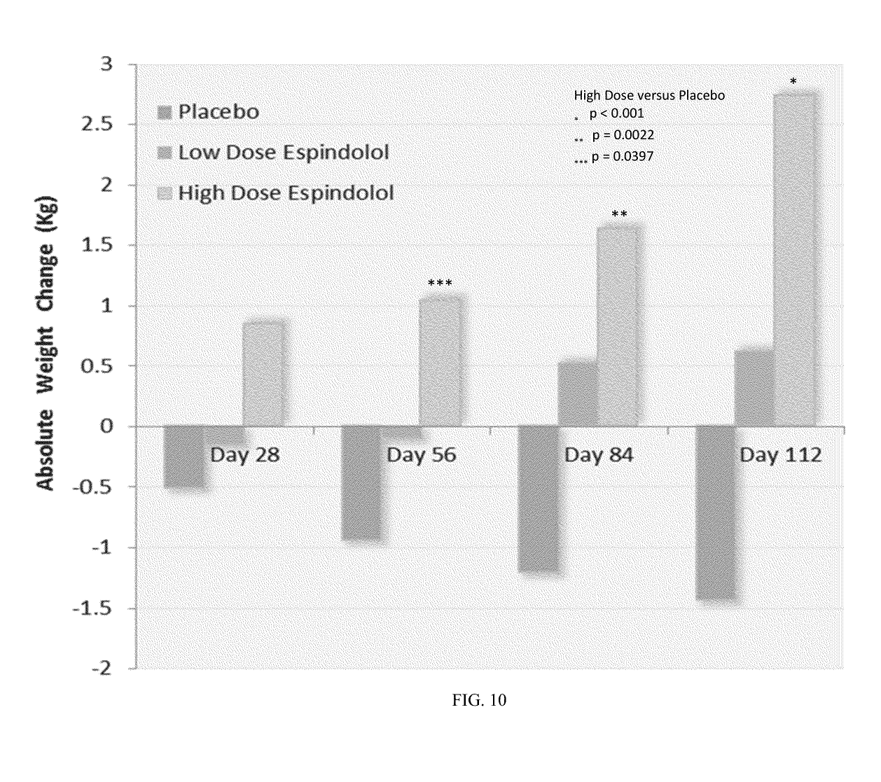

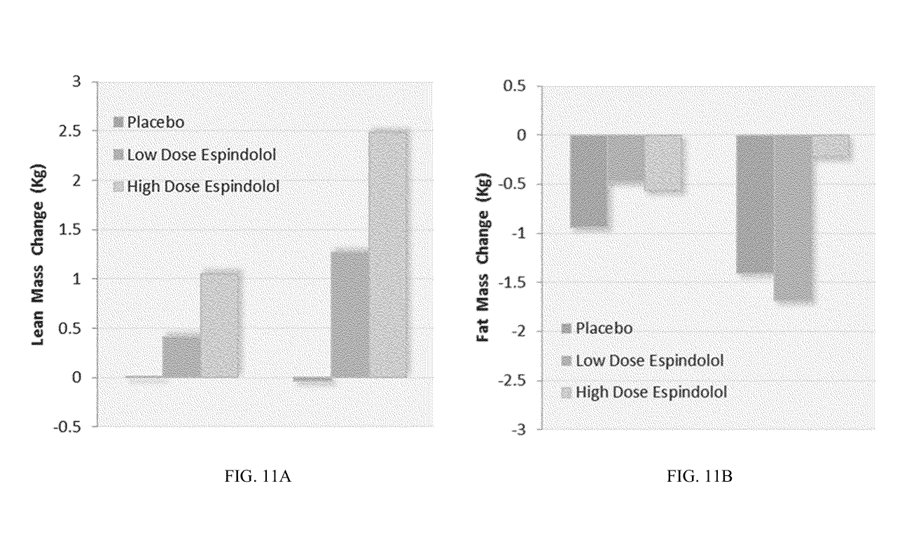

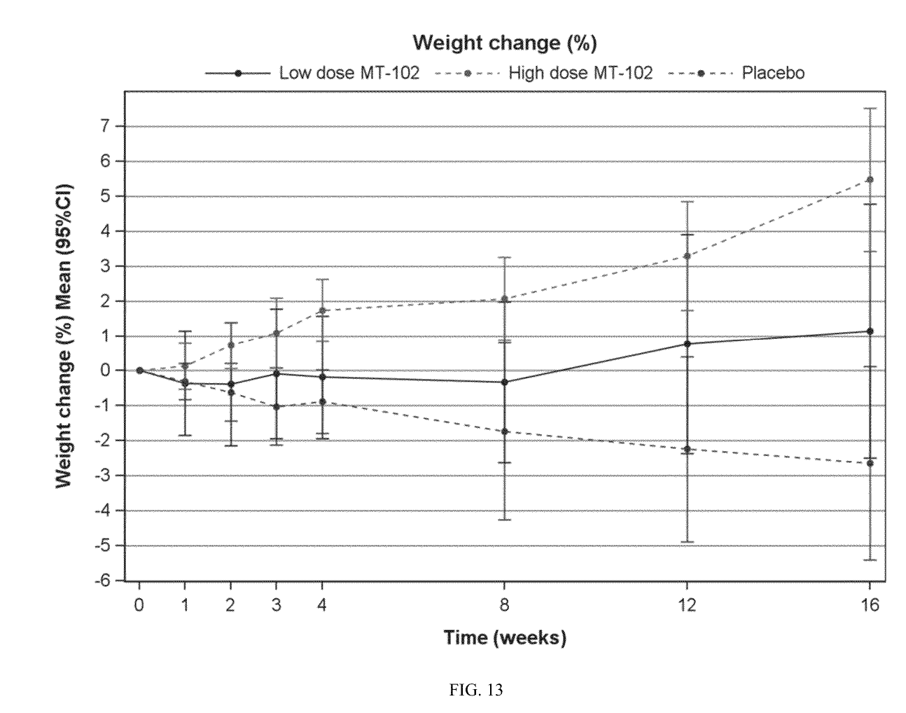

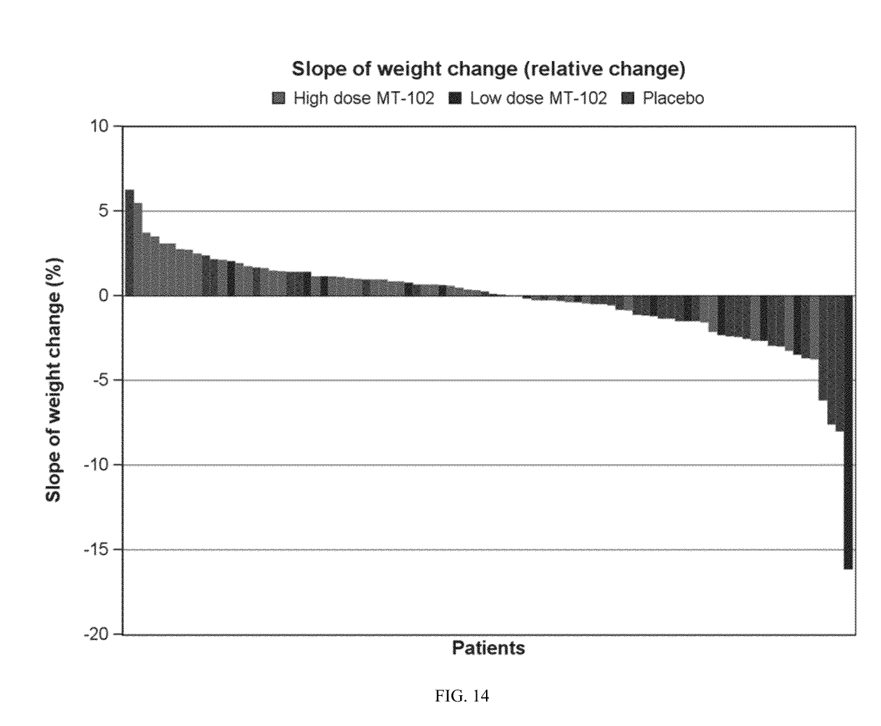

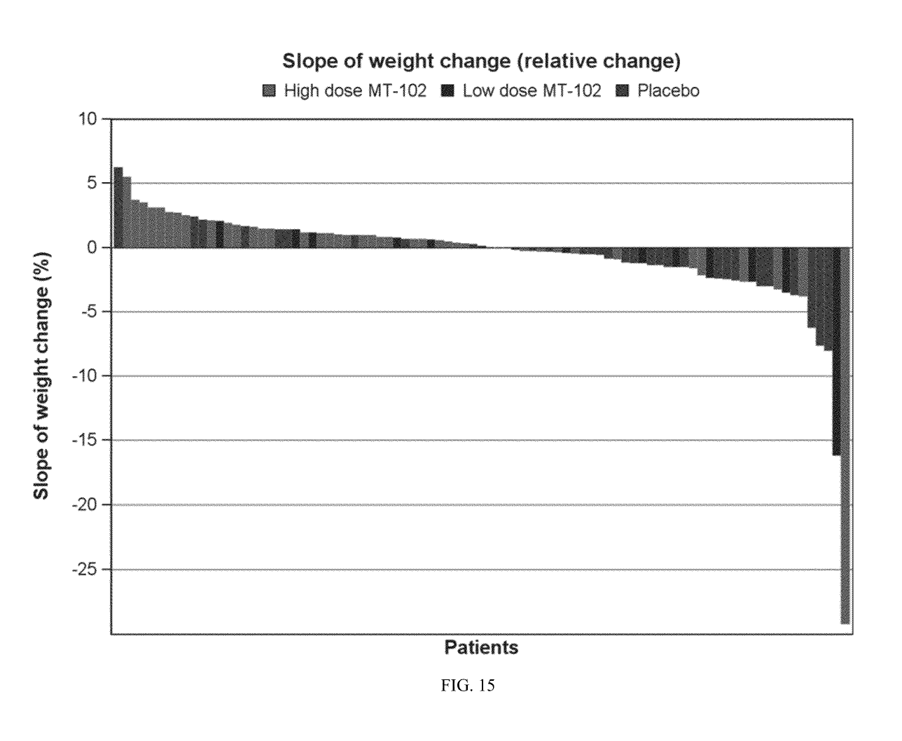

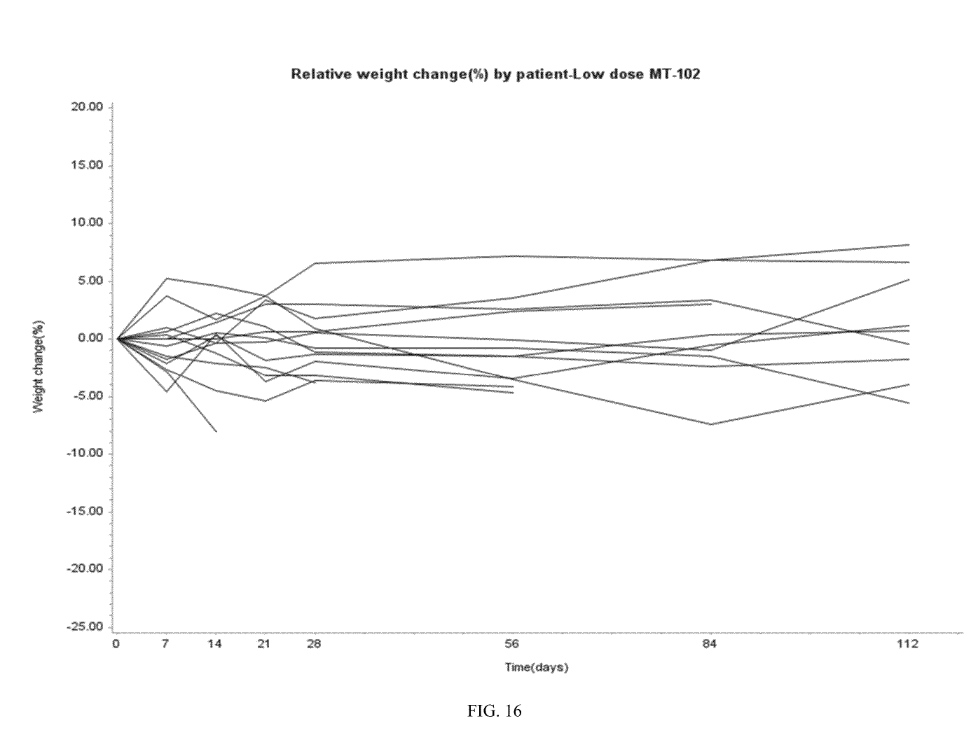

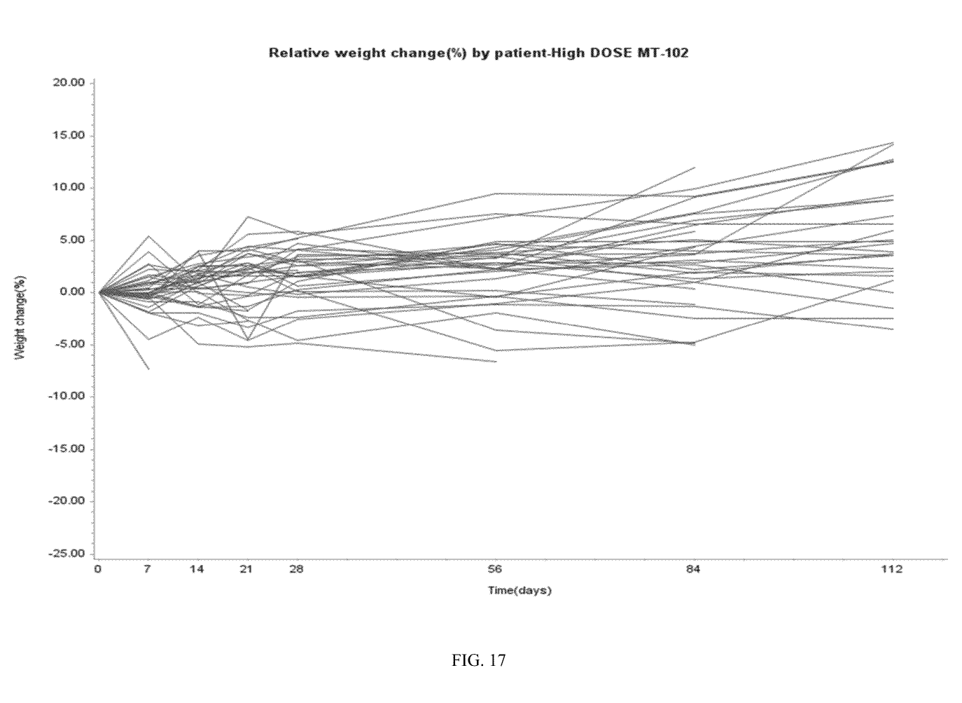

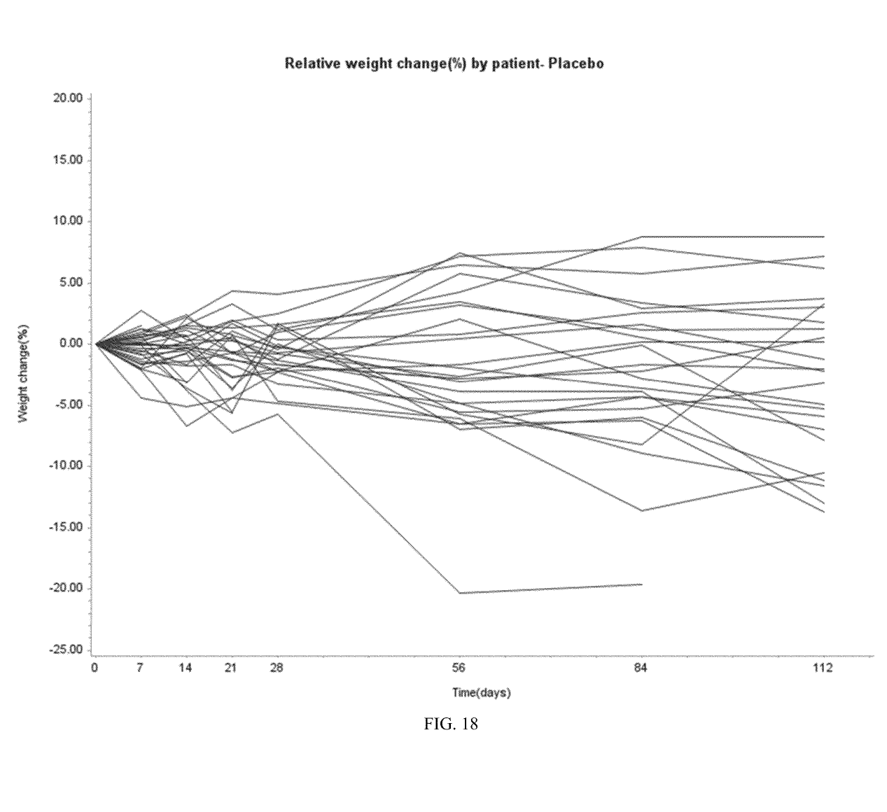

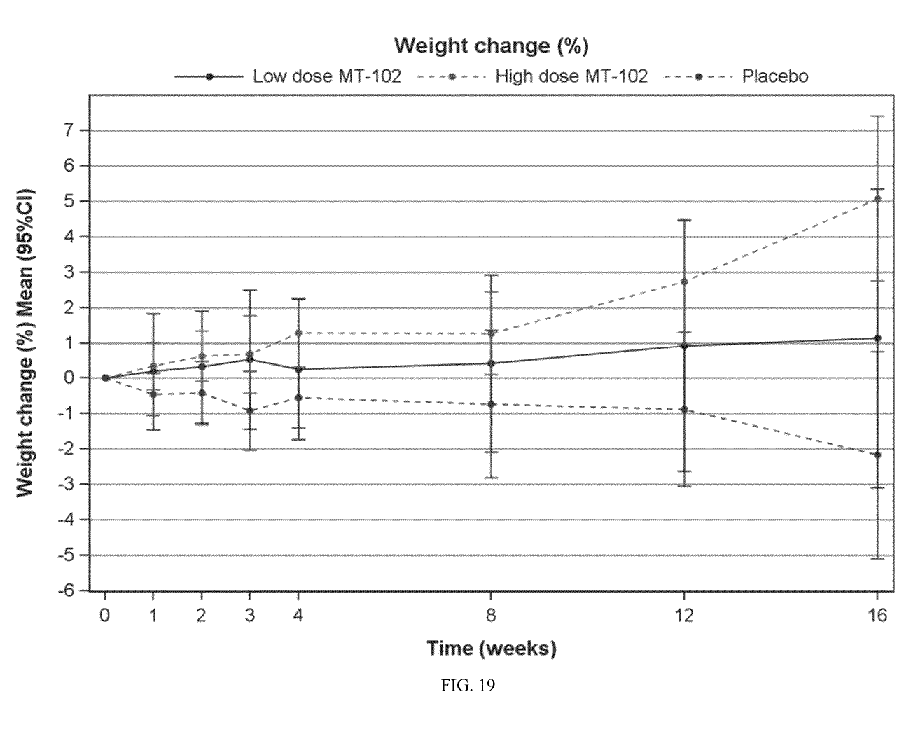

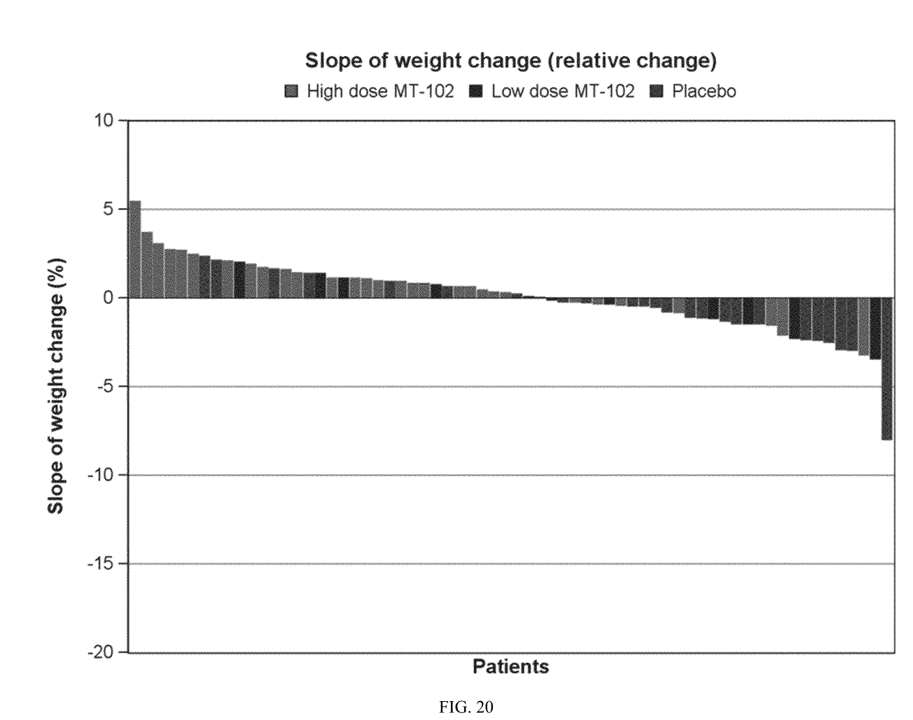

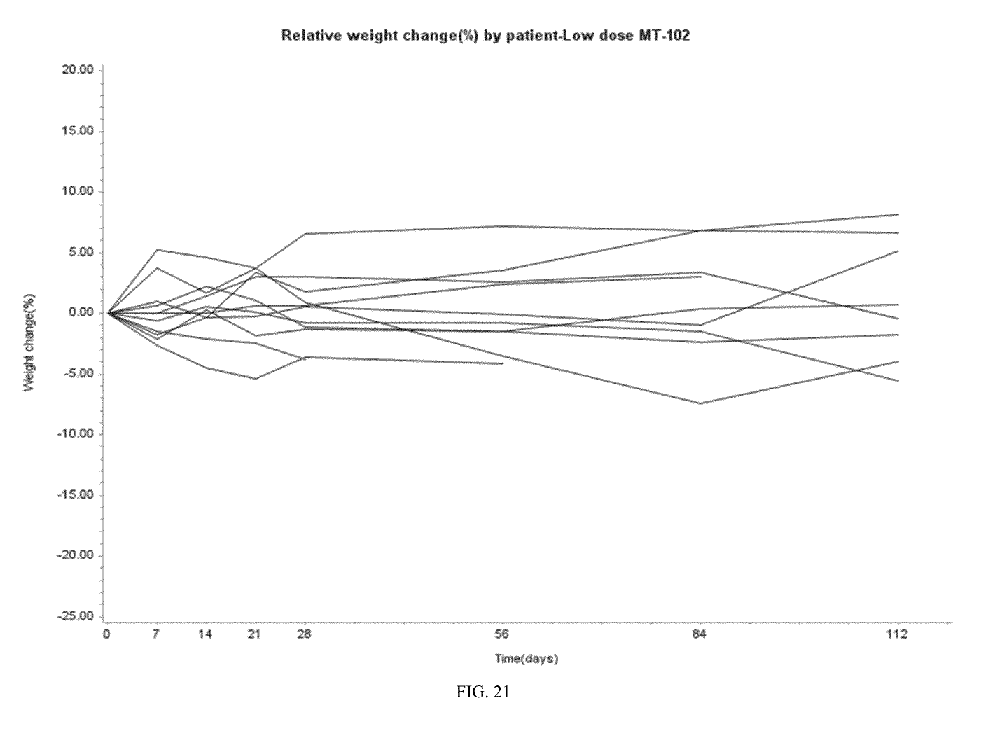

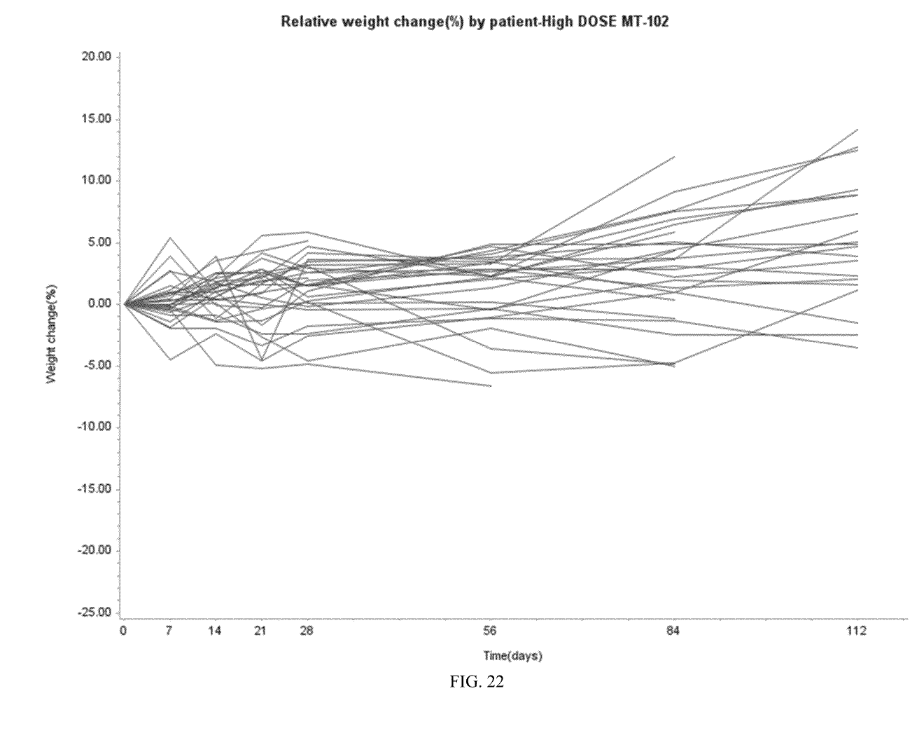

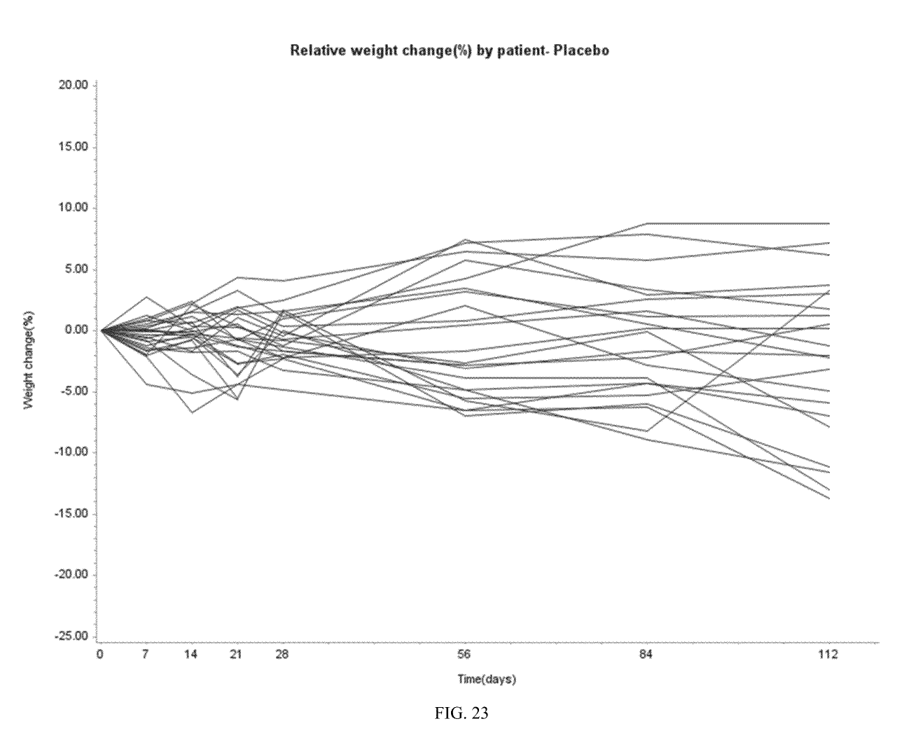

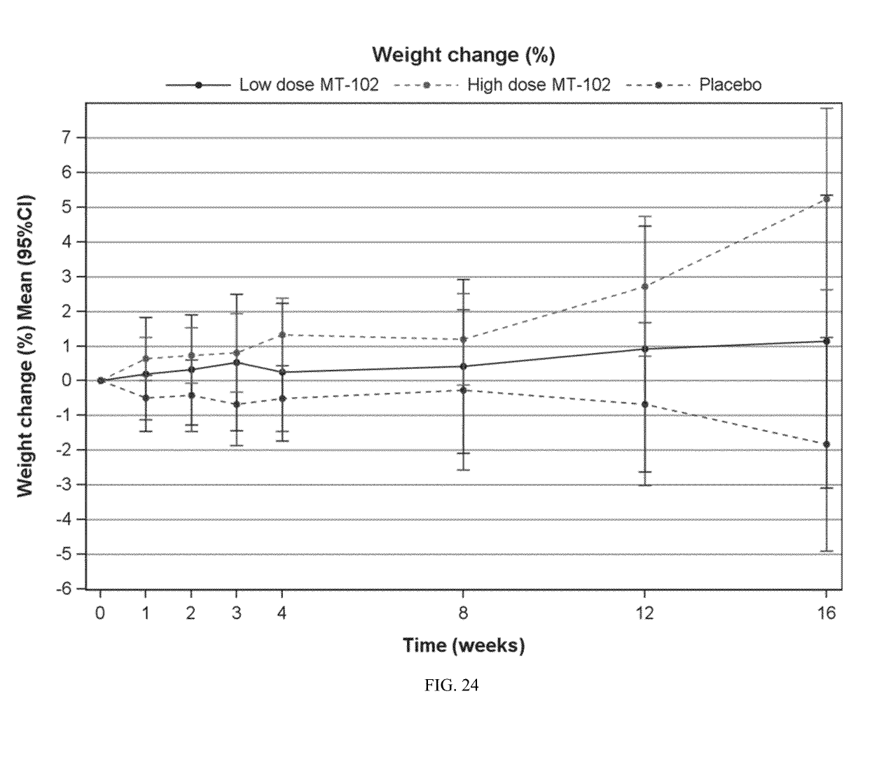

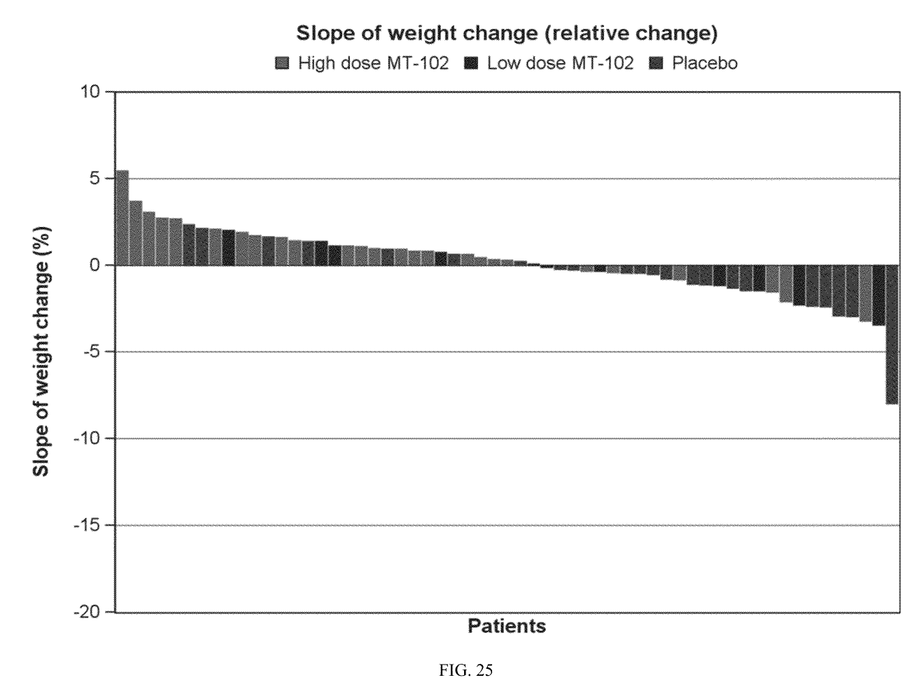

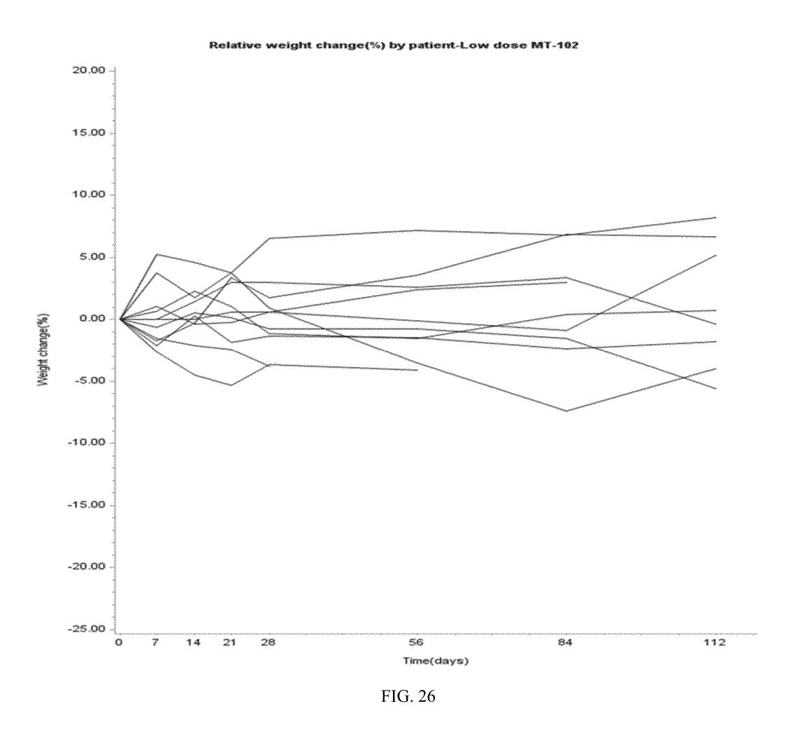

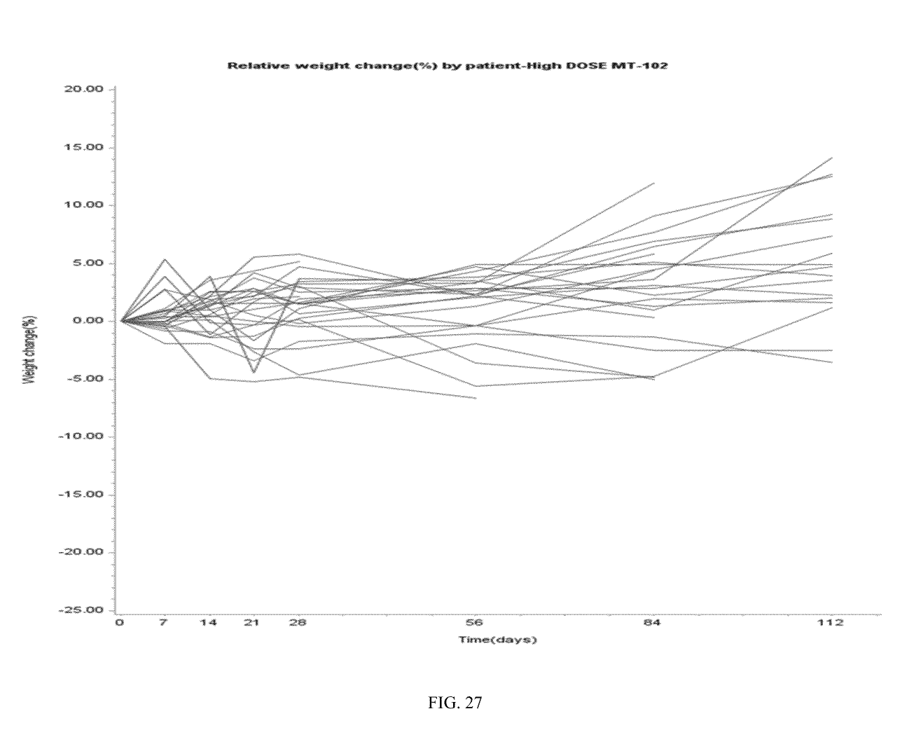

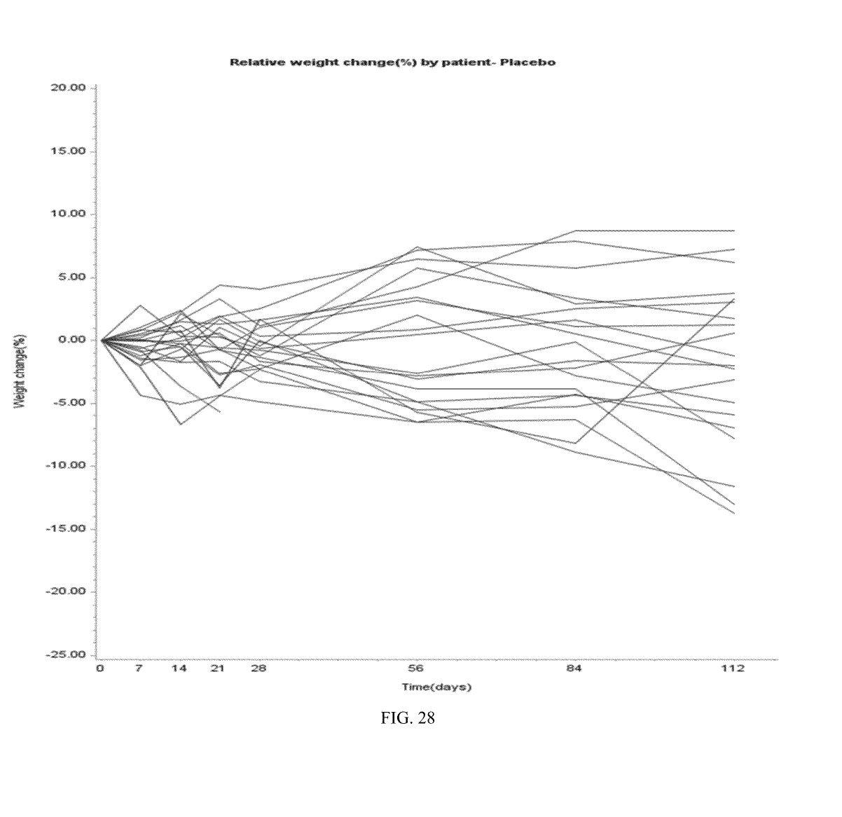

Missing data imputed using the Mixed-Effect Model for Repeated Measures (MMRM)
Assumptions for the null hypothesis for the primary endpoint:
   Two-sided significance test with an alpha of 0.05
   Power of 85%
   Expected mean weight change per 4-week period of -0.8 kg in the placebo group
   Mean weight change per 4-week period of 0 kg in the high dose MT-102 arm
   Standard deviation of 1.2 kg per 4-week period
   20% of recruited patients who do not comply with the criteria for inclusion
   Randomisation 3:2:1 (high dose / placebo / low dose)
Sample size formula for inequality tests of means in two unequal groups:
   Target recruitment of 132 patients
   Actual recruitment of 87 patients: reduced power to 78%
Safety data is shown in FIGS. 7 and 8.
Weight and body composition changes are shown in FIGS. 4, 9 to 11, 13 to 28

### CONCLUSIONS

Espindolol was well tolerated at doses of 2.5 and 10 mg bd in this population of cachectic NSCLC and CRC patients over a period of 16 weeks.

Efficacy was demonstrated for high dose (10 mg bd) espindolol compared to placebo and this effect was statistically significant in the ITT population for:
Absolute weight change (primary endpoint)
Lean mass change
Fat mass change
Hand Grip Strength change

A trend towards dose response was seen for:
Stair climbing power change
Six minute walk test change

Data from this trial support the further investigation of espindolol for the treatment of cancer cachexia at a dose of 10 mg bd.

### CITATIONS

Bassey, E J, Simple Performance Tests. In Handbook of Methods for the Measurement of Work Performance, Physical Fitness and Energy Expenditure in Tropical Populations, 1990, ed. K J Collins London: International Union of Biological Sciences, Medicinal Research Council, and London School of Hygiene and Tropical Medicine.
Bean, J F et al., Is Stair Climb Power a Clinically Relevant Measure of Leg Power Impairments in At-Risk Older Adults?, Arch Phys Med Rehabil, 2007 May; 88(5):604-9.
Enright M D, Paul L., The Six-Minute Walk Test, Respiratory Care, 2003 August; 48(8):783-5.
Guralnik, Jack M. et al., A Short Physical Performance Battery Assessing Lower Extremity Function: Association With Self-Reported Disability and Prediction of Mortality and Nursing Home Admission, J Gerontol, 1994 March; 49(2):M85-94.
NIH, National Health and Nutrition Examination Survery III: Physical Function Examination Manual, 1990, Bethesda, MD: National Institutes of Health.

1. A method of treating cachexia or sarcopenia or a combination thereof comprising administering to a human patient in need thereof a total daily oral dose of 2.5 to 20 mg of S-pindolol or a pharmaceutical formulation comprising the same.

2. The method according to claim 1, wherein the total oral dose of S-pindolol is administered bi-daily as two sub-doses.

3. The method according to claim 2, wherein each sub-dose is in the range 2.5 to 10 mg.

4. The method according to claim 1, wherein the oral formulation is a solid dose formulation.

5. The method according to claim 1, wherein the formulation comprises one or more excipients independently selected from microcrystalline cellulose, lactose, colloidal silicon dioxide, maize starch, povidone, magnesium stearate and crospovidone.

6. The method according to claim 1, wherein the formulation is a liquid.

7. The method of treating cachexia according to claim 1 wherein human patient has underlying disease selected from cancer, chronic heart failure, COPD, TB and HIV.

8. The method of treating cachexia according to claim 7, wherein the cancer is selected from colorectal cancer, lung cancer, pancreatic cancer, bone cancer, stomach cancer, oesophageal cancer, prostate cancer and ovarian cancer, for example colorectal cancer and lung cancer such as non-small cell lung cancer.

9. The method according to claim 8, wherein the S-pindolol formulation is administered pre, post and/or concomitant with a cancer treatment regime selected from surgery, chemotherapy and radiotherapy.

10. An oral pharmaceutical formulation comprising 2.5 to 20 mg of S-pindolol per dose and at least one excipient.

11. The oral pharmaceutical formulation according to claim 10 comprising 2.5 to 10 mg of S-pindolol per dose.

12. The oral pharmaceutical formulation according to claim 10, wherein the formulation is a liquid.

13. The oral pharmaceutical formulation according to claim 10, wherein the formulation is a solid dose formulation selected from a tablet and a capsule, for example for immediate release.

14. The oral pharmaceutical formulation according to claim 13, wherein one or more excipients selected from the group comprising microcrystalline cellulose, lactose, colloidal silicon dioxide, maize starch, povidone, magnesium stearate and crospovidone.

15. The oral formulation according to claim 10 wherein per dose formulated consists of: 2.5 mg S-pindolol, 3.0 mg povidone K30, 45.5 mg lactose monohydrate , 10.0 mg maize starch, 1.0 mg Aerosil 200, 17.5 mg avicel PH 101, 2.0 mg crospovidone, 17.5 mg avicel PH 102, and 1.0 mg magnesium stearate.
